(19) United States
(12) Patent Application Publication
(10) Pub. No.: US 2023/0045642 A1
(43) Pub. Date: Feb. 9, 2023

(54) S. AUREUS ANTIGENS AND COMPOSITIONS THEREOF

(71) Applicant: GLAXOSMITHKLINE BIOLOGICALS SA, Rixensart (BE)

(72) Inventors: Fabio BAGNOLI, Siena (IT); Andreas HAAG, Siena (IT); Lassi LILJEROOS, Siena (IT)

(73) Assignee: GLAXOSMITHKLINE BIOLOGICALS SA, Rixensart (BE)

(21) Appl. No.: 17/783,725

(22) PCT Filed: Dec. 15, 2020
(86) PCT No.: PCT/EP2020/086155

§ 371 Date: Jun. 9, 2022

(30) Foreign Application Priority Data
Dec. 19, 2019 (EP) 19218329.1

(51) Int. Cl. A61K 39/085; C07K 14/31; A61P 31/04

(52) U.S. Cl. CPC A61K 39/085; C07K 14/31; A61P 31/04

(57) ABSTRACT

The invention provides improved staphylococcal antigens. Immunogenic compositions are also provided. The invention may find use in the prevention and treatment of staphylococcal infections.

### FIELD OF THE INVENTION

The invention relates to antigens derived from Staphylococcus aureus and to their use in immunisation.

### BACKGROUND TO THE INVENTION

Staphylococcus aureus (S. aureus) is a Gram-positive commensal bacterium which colonises the skin, nares, axilla, and pharynx of up to 30% of human subjects. S. aureus is also a major bacterial pathogen causing significant disease burden in both hospital and community settings. S. aureus causes a range of illnesses from minor skin infections to life-threatening diseases such as pneumonia, meningitidis, osteomyelitis, bacteraemia, endocarditis, toxic shock syndrome, organ abscesses and septicaemia.

Bacterial pathogenicity results from the expression of multiple virulence factors which are differently expressed during different phases of S. aureus lifecycle. Additionally, S. aureus exhibits adaptability to adverse stimuli and environmental conditions by rapid exchange and/or acquisition of DNA and by the mutability of its genome (Alonzo F 3rd, Torres V J, Microbiol Mol Biol Rev. 2014 June; 78(2):199-230).

A remarkable pathogenic attribute of S. aureus is the diverse repertoire of immune system evasion factors expressed, which have evolved to dampen the host immune system, thus inhibiting the clearance of the pathogen and preventing the development of immunological memory against it.

Among staphylococcal immune system evasion factors, the role of factors that prevent immune cell recognition and killing (protein A, catalase), cytotoxins (hemolysins, cytolytic peptides, leukocidins), and immunomodulatory proteins (superantigens, complement inhibitory proteins) in infection is acknowledged.

The clinical standard of care for invasive S. aureus infections includes aggressive administration of antibiotics. However, the recent increase in the incidence of multidrug-resistant isolates and the increase dominance of highly virulent clonal lineages have diminished the success of such therapeutic strategies (Alonzo F 3rd, Torres V J, Microbiol Mol Biol Rev. 2014 June; 78(2):199-230).

Passive immunotherapy involving administration of polyclonal antisera and monoclonal antibodies against staphylococcal antigens represents an alternative to antibiotic treatment; however, several passive immunisation candidates have failed to show efficacy in humans.

An alternative approach is to use active vaccination to generate a polyclonal response against S. aureus. One of the limitations of staphylococcal vaccine candidates is the complexity of the bacterial immune evasion factors expressed by the pathogen, which have adapted to avoid immune recognition strategies. Targeting immune evasion factors as antigens useful for a staphylococcal vaccine should promote natural clearance of the pathogen.

Among staphylococcal immune evasion factors, leukocidins are toxins that are able to target numerous immune and non-immune cells (Alonzo F 3rd, Torres V J, Microbiol Mol Biol Rev. 2014 June; 78(2):199-230). They consist of two separate water-soluble monomeric subunits called leukocidin components, each comprising a cap, a rim and a stem domain. Leukocidin components, upon dimerization, form a beta-barrel pore spanning the host target cell's phospholipidic bilayer.

Despite leukocidins representing promising vaccine candidates, their use has been restrained by their inherent toxicity.

Thus there remains a need to identify improved leukocidin components for use in S. aureus vaccines especially in view of increasing frequency of multidrug resistant strains.

### SUMMARY OF THE INVENTION

The inventors have surprisingly found that modified leukocidin components comprising a deletion within the stem domain are able to self-assemble but their toxicity is reduced. Accordingly, the present invention provides modified leukocidin components, complexes and immunogenic compositions thereof which can be useful to induce protective immunity against S. aureus.

One aspect of the invention provides a modified Staphylococcal leukocidin component polypeptide in which the stem domain has been fully or partially deleted, and wherein the unmodified leukocidin component polypeptide comprises or consists of an amino acid sequence having at least 80%, 85%, 90%, 92%, 95%, 96%, 97%, 98%, 99%, or 100% sequence identity to:
a polypeptide comprising the amino acid sequence of SEQ ID NO.: 1; or
a polypeptide comprising the amino acid sequence of SEQ ID NO.: 4.

The leukocidin component of the invention is suitably a S. aureus antigen.

In one embodiment the unmodified leukocidin component polypeptide is SEQ ID NO.: 1, and one or more of amino acids E107 to Q146 of SEQ ID NO.: 1 are deleted. In one embodiment the unmodified leukocidin component polypeptide is SEQ ID NO.: 4 and one or more of amino acids T104 to K142 of SEQ ID NO.: 4 are deleted.

In one embodiment, at least 5, 10, 15, 20, 30, 40, 50 60, 70 or 90 amino acids are deleted. In one embodiment, the at least 5 amino acid which are deleted are consecutive. In one embodiment, additional 1 to 10 amino acids flanking the N-terminus and/or at the C-terminus of the stem domain of the unmodified leucocidin polypeptide are deleted, for example, 1, 2, 3, 4, 5, 6, 7, 8, 9 or 10 amino acids flanking the N-terminus and/or at the C-terminus of the stem domain. In a preferred embodiment, where the unmodified leukocidin component polypeptide is SEQ ID NO.: 1, residue Y116 of SEQ ID NO.: 1 is deleted, and where the unmodified leukocidin component polypeptide is SEQ ID NO.: 4, residue Y113 of SEQ ID NO.: 4 is deleted.

In a preferred embodiment, the polypeptide the invention comprises or consists of an amino acid sequence having at least 80%, 85%, 90%, 92%, 95%, 96%, 97%, 98%, 99%, or 100% sequence identity to SEQ ID NO.: 2 or SEQ ID NO.: 5.

In one embodiment, the amino acid sequence which is deleted within the stem domain is replaced by a linker. In one embodiment, the linker comprises or consists of a sequence of SEQ ID NO.: 7.

In one embodiment, the polypeptide of the invention comprises an amino acid sequence having at least 80%, 85%, 90%, 92%, 95%, 96%, 97%, 98%, 99%, or 100% sequence identity to SEQ ID NO.: 3 or SEQ ID NO.: 6.

In one embodiment, the modified leukocidin component of the invention is detoxified.

A further aspect of the invention provides a polynucleotide encoding the polypeptide of the invention. Vectors comprising such a polynucleotide are also provided. Also provided is a host cell comprising a polynucleotide or vector of the invention.

It is also provided a complex comprising a modified leukocidin component of the invention. In one embodiment, the invention provides a complex comprising a polypeptide comprising an amino acid sequence having at least 80%, 85%, 90%, 92%, 95%, 96%, 97%, 98%, 99%, or 100% sequence identity to SEQ ID NO.: 3, and/or a polypeptide comprising an amino acid sequence having at least 80%, 85%, 90%, 92%, 95%, 96%, 97%, 98%, 99%, or 100% sequence identity to SEQ ID NO.: 5. In one embodiment, the complex of the invention is detoxified.

In one aspect, it is provided an immunogenic composition comprising the polypeptide or the complex of the invention. In one embodiment, the immunogenic composition of the invention comprises additional antigens. In a preferred embodiment, the immunogenic composition comprises a ClfA antigen, an Hla antigen, or an SpA antigen, and a staphylococcal capsular polysaccharide.

In one embodiment, the capsular polysaccharide is a S. aureus serotype 5 and/or type 8 capsular polysaccharide. In a preferred embodiment, the immunogenic composition of the invention comprises a ClfA antigen comprising an amino acid sequence having at least 80%, 85%, 90%, 92%, 95%, 96%, 97%, 98%, 99%, or 100% sequence identity to SEQ ID NO: 8, a Hla antigen comprising the amino acid sequence having at least 80%, 85%, 90%, 92%, 95%, 96%, 97%, 98%, 99%, or 100% sequence identity to SEQ ID NO: 9, a SpA antigen comprising the amino acid sequence having at least 80%, 85%, 90%, 92%, 95%, 96%, 97%, 98%, 99%, or 100% sequence identity to SEQ ID NO: 10, a S. aureus serotype 5 capsular polysaccharide, and a S. aureus serotype type 8 capsular polysaccharide.

In one embodiment, the capsular polysaccharide is conjugated to a carrier protein. In one embodiment, the capsular polysaccharide is conjugated to one of a ClfA antigen, a Hla antigen, or a SpA antigen. In a preferred embodiment, the immunogenic composition comprises a S. aureus serotype 5 capsular polysaccharide conjugated to a Hla antigen and/or a type 8 capsular polysaccharide conjugated to a ClfA antigen. In a preferred embodiment, said ClfA-type 8 capsular polysaccharide and Hla-serotype 5 capsular polysaccharide conjugates are bioconjugates.

The invention provides an adjuvanted immunogenic composition. In one embodiment, the adjuvant comprises a saponin and a lipopolysaccharide. In one embodiment, the adjuvant comprises a saponin and a lipopolysaccharide in a liposomal formation. In one embodiment, the saponin is an immunologically active saponin fraction derived from the bark of Quillaja Saponaria Molina. In a preferred embodiment, the saponin is QS21. In one embodiment, the lipopolysaccharide is a lipid A derivative. In a preferred embodiment, the lipopolysaccharide is 3D-MPL. In one embodiment, the immunogenic composition further comprises a sterol. In one preferred embodiment, the sterol is cholesterol.

In one aspect, it is provided a vaccine comprising the immunogenic composition of the invention.

In one aspect, the invention provides a method for the treatment or prevention of staphylococcal infection, for example S. aureus infection, in a subject in need thereof comprising administering to said subject a therapeutically effective amount of the polypeptide of the invention, the complex of the invention, the immunogenic composition of the invention, or the vaccine of the invention. The subject is preferably a mammal, and more preferably a human.

### DESCRIPTION OF THE FIGURES

FIG. 1: In vitro expression of lukD (panel A) and lukE (panel B). S. aureus strain Newman mRNA was extracted from cultures harvested at OD₆₀₀=0.5 (early-log), 2 (mid log phase), 4 (late-exponential), 8 (pre-stationary), and 12 (stationary phase). cDNA was synthetized, and qPCR was performed on cDNA derived from 25 ng of RNA before reverse transcription. Samples were normalized to the expression levels of the housekeeping gene gyrB and relative expression values to the reference culture OD₆₀₀=2 (mid log phase) were calculated using the ΔΔct method for OD 0.5 (early-log), 4 (late-exponential), 8 (pre-stationary) and 12 (stationary phase).

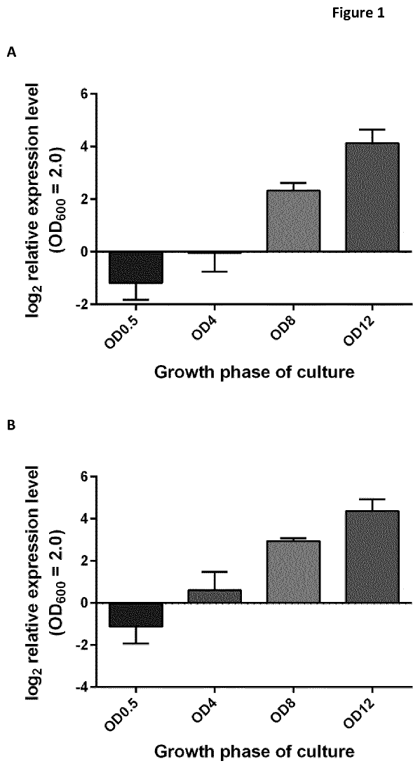

FIG. 2: In vivo expression of lukD and lukE. eight to ten-weeks old female CD1 mice (pathogen free) were infected intravenously with a sub-lethal dose of S. aureus (˜1×107 CFU per mouse) and organs harvested at defined time points. 250 ng of host extracted mixed cDNA samples. S. aureus cultures used as the inoculum prior to infection (OD₆₀₀=2.0) served as baseline for bacterial gene expression levels and cDNA prepared from uninfected mouse kidneys served as control for background and unspecific amplification, respectively.

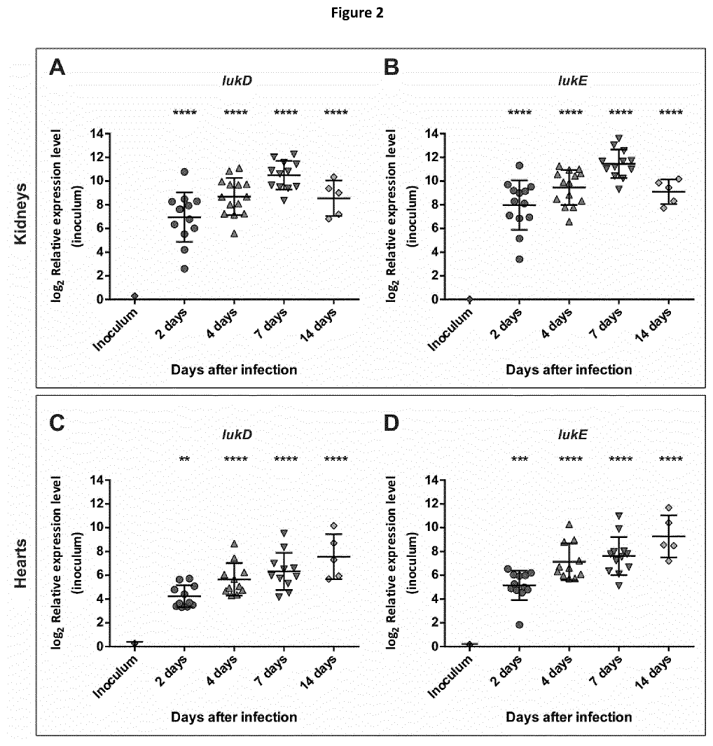

FIG. 3: Antigen design. Schematic representation of the (from top to bottom) wild type lukED locus for recombinant antigen production: single lukE and lukD expression constructs for wild type protein expression as well as expression of stemless versions of LukE and LukD, respectively. Functional domains are indicated. Bottom shows schematic representation of the oligomeric toxins formed in the wild-type cytotoxic LukED containing the pore-forming stem region and oligomeric engineered LukED missing the stem region.

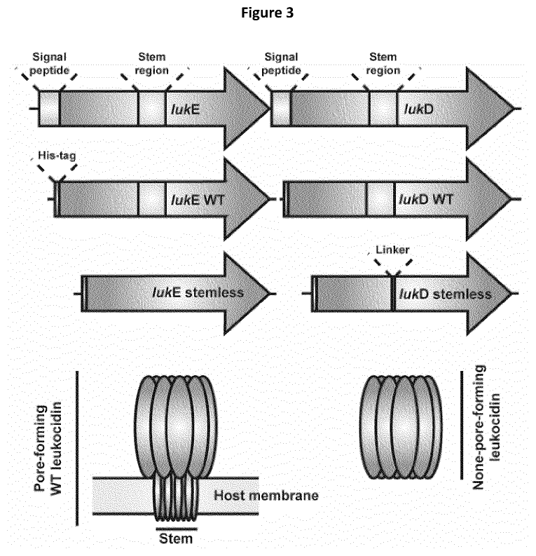

FIG. 4: Enhanced stability of Astern complex compared to WT or Astern monomers. The thermal stability of purified recombinant LukE and LukD stemless protein variants (panel A) and LukE and LukD WT variants (panel B) was assessed by differential scanning calorimetry (DSC).

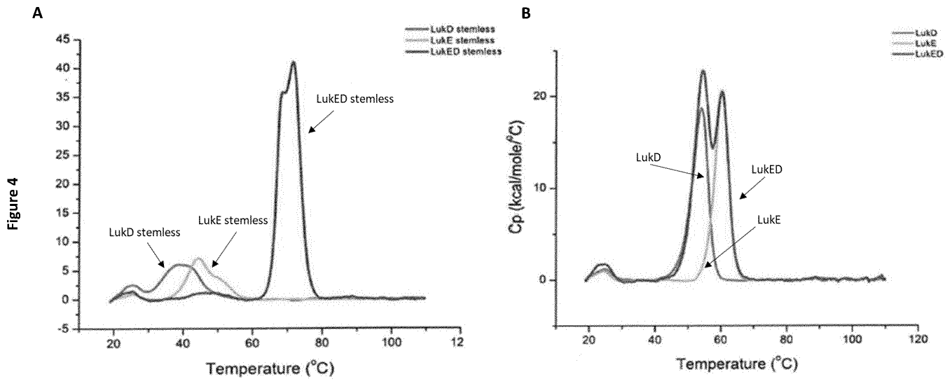

FIG. 5: Detoxification of LukED mutants. Cytotoxicity of the indicated LukED variants was assessed on the THP-1 cell line (ATCC: TIB-202).

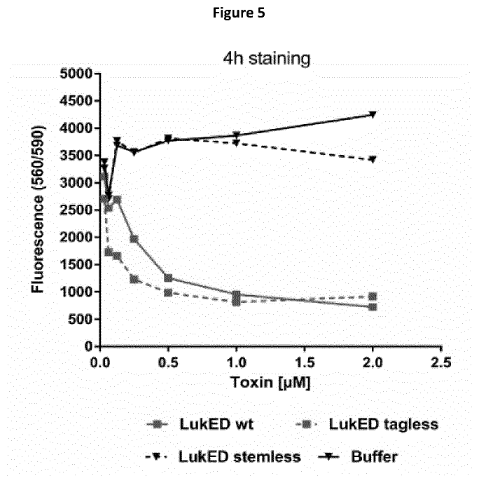

FIG. 6: Immunisation with Luk mutants in protective in skin infection model. Ability of vaccination with the indicated LukED versions to protect mice infected with S. aureus strain USA300-LAC by reducing size of skin lesions (A) and number of viable bacteria recovered from skin biopsies (B).

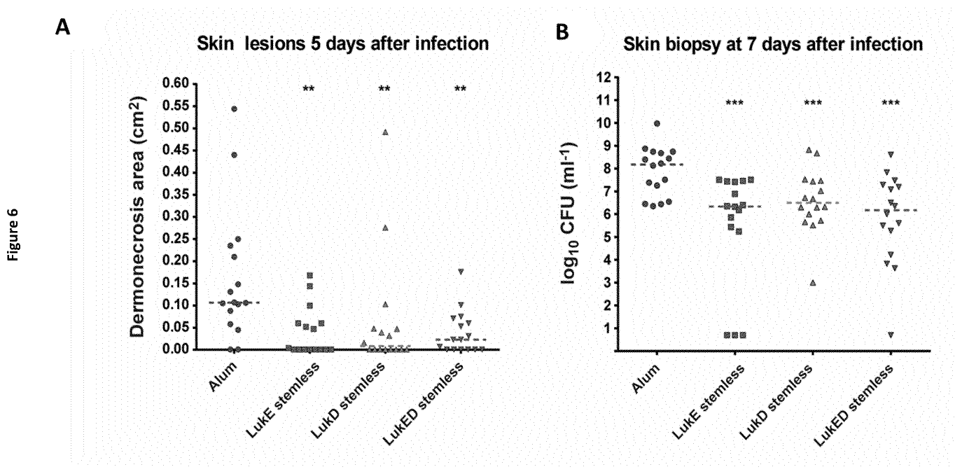

### DETAILED DESCRIPTION OF THE INVENTION

### General

The term “comprising” encompasses “including” as well as “consisting of” e.g. a composition “comprising” X may consist exclusively of X or may include something additional e.g. X+Y. References to “comprising” (or “comprises”, etc.) may optionally be replaced by references to “consisting of” (or “consists of”, etc.). The term “consisting essentially of” limits the scope of a claim to the specified materials or steps “and those that do not materially affect the basic and novel characteristic(s)” of the claimed invention.

The term “about” in relation to a numerical value x is optional and means, for example, x±10%.

As used herein, references to “percentage sequence identity” between a query amino acid sequence and a subject amino acid sequence are understood to refer to the value of identity that is calculated using a suitable algorithm or software program known in the art to perform pairwise sequence alignment.

A query amino acid sequence may be described by an amino acid sequence identified in one or more claims herein. The query sequence may be 100% identical to the subject sequence, or it may include up to a certain integer number of amino acid alterations (e.g. point mutations, substitutions, deletions, insertions etc.) as compared to the subject sequence, such that the % identity is less than 100%. For example, the query sequence is at least 80, 85, 90, 95, 96, 97, 98, or 99% identical to the subject sequence.

In order to calculate percent identity, the query and subject sequences may be compared and aligned for maximum correspondence over a designated region (e.g. a region of at least about 40, 45, 50, 55, 60, 65 or more amino acids in length, and can be up to the full length of the subject amino acid sequence).

Any N-terminal or C-terminal amino acid stretches that may be present in the query sequence, such as signal peptides or leader peptide or C-terminal or N-terminal tags, should excluded from the alignment.

Alternatively, percentage sequence identity may be calculated over the “full length” of the subject sequence.

Alignment may be determined by the Smith-Waterman local alignment algorithm which is disclosed in Smith & Waterman (1981) Adv. Appl. Math. 2: 482-489 and is implemented in the publicly available local alignment tool EMBOSS Water (https://www.ebLac.uk/Tools/psa/). The alignment may be determined using an affine gap search with a gap open penalty of 10 and a gap extension penalty of 0.5, BLOSUM matrix of 62. An alternative or additional local alignment tool implementing the Smith-Waterman local alignment algorithm is JAligner. An alternative or additional local alignment tools is EMBOSS Matcher. Alternative or additional local alignment tools are software implementing Basic Local Alignment Search Tool (BLAST) algorithms, which are publicly available through the National Centre for Biotechnology Information (www.ncbi.nlm.nih.gov).

Alternatively or additionally, alignment may be determined by the Needleman-Wunsch global alignment algorithm which is disclosed in Needleman & Wunsch (1970) J. Mol. Biol. 48, 443-453 and is implemented in the publicly available global alignment tool EMBOSS Needle (https://www.ebLac.uk/Tools/psa/). The alignment may be determined with a gap open penalty of 10 and a gap extension penalty of 0.5, BLOSUM matrix of 62. An alternative or additional global alignment tool implementing the Needleman-Wunsch global alignment algorithm is EMBOSS Stretcher.

As used herein, the term “fragment” is a portion of a protein smaller than the whole. As used herein, the term “immunogenic fragment” is a portion of an antigen smaller than the whole, that is capable of eliciting a humoral and/or cellular immune response in a host animal, e.g. a human, specific for that fragment.

Fragments of a protein can be produced using techniques known in the art, e.g. recombinantly, by proteolytic digestion, or by chemical synthesis. Internal or terminal fragments of a polypeptide can be generated by removing one or more nucleotides from one end (for a terminal fragment) or both ends (for an internal fragment) of a nucleic acid which encodes the polypeptide. Typically, fragments comprise at least 10, 20, 30, 40, or 50 contiguous amino acids of the full-length sequence. However, fragments may also be 100 or more or 200 or more amino acids in length. Fragments may be readily modified by adding or removing 1, 2, 3, 4, 5, 6, 7, 8, 9, 10, 20, 30, 40 or 50 amino acids from either or both of the N and C termini.

As used herein the term “leukocidin component” refers to a subunit of a leukocidin, comprising a cap, a rim and a stem functional domains. As used herein the term “modified leukocidin component” refers to a genetically modified leucocidin component, such as a leukocidin component comprising a deletion. A modified leukocidin component may lack all or parts of at least the cap, rim and stem domains. Said modifications of a modified leukocidin component (e.g., deletion) are respective to an unmodified leukocidin component. As used herein the term “unmodified leukocidin component” refers to leukocidin component which is not genetically modified, such as a polypeptide comprising or consisting of the amino acid sequence of SEQ ID NO.: 1; or a polypeptide comprising or consisting of the amino acid sequence of SEQ ID NO.: 4.

As used herein, the term leukocidin component can be shortened as Luk. As used herein, the term leukocidin D can be shortened as LukD. As used herein, the term leukocidin E can be shortened as LukE.

As used herein the term “leukocidin” or “leukocidin complex” refers to an oligomeric complex comprising leukocidin components. As used herein, the term LukED refers to a complex comprising LukE and LukD. The subunits of the complex may suitably be in equimolar ratio.

As used herein, the term ClfA refers to Clumping factor A from S. aureus. As used herein, the term Hla refers to Alpha-haemolysin, also known as alpha-toxin, from S. aureus. As used herein, the term SpA refers to Staphylococcal protein A from S. aureus. As used herein, the term Capsular polysaccharide can be shortened as CP.

As used herein, the term “carrier protein” refers to a protein covalently attached to an antigen (e.g. saccharide antigen) to create a conjugate (e.g. bioconjugate). A carrier protein activates T-cell mediated immunity in relation to the antigen to which it is conjugated.

As used herein, the term “conjugate” refers to a saccharide (such as a capsular polysaccharide) covalently linked to a carrier protein.

As used herein, the term “bioconjugate” refers to conjugate between a protein (e.g. a carrier protein) and an antigen (e.g. a saccharide) prepared in a host cell background, wherein host cell machinery links the antigen to the protein (e.g. N-links).

As used herein, the term “subject” refers to an animal, in particular a mammal such as a primate (e.g. human).

As used herein, the term “deletion” is the removal of one or more amino acid residues from the amino acid sequence of a protein.

As used herein, the term “insertion” is the addition of one or more non-native amino acid residues in the amino acid sequence of a protein.

As used herein, reference to “between amino acids . . . ” (for example “between amino acids G79 and H169”) is referring to the amino acid number counting consecutively from the N-terminus of the amino acid sequence, for example “between amino acids G79 and H169 of SEQ ID NO.: 1 refers to any position in the amino acid sequence between the 79th and the 169th amino acid, inclusive of the endpoints of SEQ ID NO.: 1.

### STATEMENT OF THE INVENTION

Leukocidins (also referred to as bicomponent leukotoxins) are complexes comprising multiple copies of two subunits, the “S” (slow-eluting) and “F” (fast-eluting) components. S components include LukS-PV, LukM, HlgA (γ-hemolysin), HlgC (gamma-hemolysin), LukE, and LukS-I. F components include LukF-PV, LukF′-PV, HlgB (γ-hemolysin), LukD, and LukF-I. The present invention is directed to LukE and LukD.

S. aureus strains can produce several leukocidins, including: Panton-Valentine leukocidin (PVL or LukFS-PV), leukocidin E/D (LukED), γ-hemolysin (HlgAB and HlgCB), and leukocidin A/B (LukAB; also known as LukHG). Of those, all strains of S. aureus are capable of producing at least three leukocidins (HlgAB, HlgCB and LukAB/HG), while most highly virulent clinical isolates can produce five leukocidins (HlgAB, HlgCB, LukAB/HG, PVL and LukED) (Alonzo F 3rd, Torres V J, Microbiol Mol Biol Rev. 2014 June; 78(2):199-230).

In general, Leukocidins are oligomeric complexes of four S component subunits and four F component subunits, which may be toxic to host target cells due to their ability to form octameric pores in the host cells' membrane.

Crystallographic studies described the main structural features of leukocidins. Functional domains of the individual S and F components include a cap, a rim and a stem. The rim domain comprises aromatic residues that are able to recognize and bind to phospholipids and other molecules. The stem domain is a glycine-rich, hydrophobic domain, which undergoes dramatic structural changes to promote the stem insertion into the membrane of host cells, forming a presumed beta-barrel pore-like structure that ultimately perforates the membrane. The cap consists of the beta-sandwich and the latch domain: the beta-sandwich domain contains key residues for inter-subunit interactions and the amino latch is believed to be involved in stem positioning during the monomeric-to-oligomeric pore transition for alpha-hemolysin, although the function may not be conserved in all the leukocidins (Nocadello S et al., Acta Crystallogr D Struct Biol. 2016 January; 72(Pt 1):113-20).

The proposed model for leukocidin action on host cells is based on the reciprocal recognition of the S or F components, which is preceded by the recognition of proteins and/or lipids on the host surface in a species- and cell type-specific manner. In general, the S component is responsible for cell recognition and for the F component recruitment, although the opposite case is also possible. Subsequently, subunit oligomerization into an octameric structure of alternating S and F subunits forms a pre-pore. Dramatic structural changes to the pre-pore lead to the stem domain insertion into the host cell membrane. The S and F component can be defined as the protomers (i.e. the structural units) of the oligomeric pore. The octameric pore is expected to be the most stable conformation for all leukocidin complexes.

Unlike other leukocidins, including LukAB/HG, LukE and LukD exhibit little to no sequence diversity among sequences of S. aureus strain and their overall frequency is estimated to be approximatively 70%, with strict lineage dependence.

LukED exhibits broad toxic activity on a variety of cell types from various species, including murine cells, rabbit and human immune cells, including neutrophils, T cells, macrophages, NK cells and dendritic cells (Alonzo F 3rd, Torres V J, Microbiol Mol Biol Rev. 2014 June; 78(2):199-230). Injection into the skin of rabbits of purified recombinant LukED elicits inflammation and dermonecrosis and in murine models of systematic infection contributes significant to animal mortality. LukED is directly lytic to infiltrating phagocytic leukocytes in vivo, as evidenced by decreased cellular viability measured by flow cytometry.

Interaction with target cell components may dictate the leukocidins' cellular tropism. Accordingly, LukED has been found to target CCR5 in macrophages, T cells and dendritic cells, via initial binding of the S component (LukE). In addition, LukED targets CXCR1/2 on primary human neutrophils, monocytes, NK cells, and a subset of CD8+ cells. Thus, LukED can facilitate the disarming of both the innate and adaptive immune system.

In the NCTC 8325 strain LukD has amino acid sequence SEQ ID NO.: 1 and LukE has amino acid sequence of SEQ ID NO.: 4.

The interaction of LukED with its target ligand CXCR1 and CXCR2 occurs though the interaction of loop L3, corresponding to amino acids Q182-A191 of SEQ ID NO.: 4. Interestingly, the alignment of the sequences of LukE from 150 different strains did not identify relevant variability in loop L3 (Nocadello S et al., Acta Crystallogr D Struct Biol. 2016 January; 72(Pt 1):113-20). Another region important for the toxic activity of LukED is the region called DR5, which corresponds to amino acids L237-R267 of SEQ ID NO.: 4.

The residues involved in the interactions occurring at the two interfaces between the protomers have been identified, wherein interface 1 corresponds to the interface between LukE and LukD and interface 2 corresponds to the interface between LukD and LukE.

Amino acids D43 and D49 of SEQ ID NO.: 1 and K15 and R16 of SEQ ID NO.: 4 are involved inter-protomer electrostatic interactions in interface 1 of the cap domain; R150 of SEQ ID NO.: 1 and D198 of SEQ ID NO.: 4 are also involved in electrostatic interaction in interface 1 of the rim domain. Additionally, D38 and E147 of SEQ ID NO.: 4 and K20 and R218 of SEQ ID NO.: 1 are involved in the interprotomer electrostatic interaction on interface 2.

Moreover, D38 of SEQ ID NO.: 4 and D43 of SEQ ID NO.: 1 seem to also be important in stabilizing the stems in the soluble form, as they are involved in intra-protomer hydrogen bond interaction with amino acids Y113 of SEQ ID NO.: 4 and Y116 of SEQ ID NO.: 1. Besides the hydrogen bonds that occur in the interprotomer-sheets, two ion pairs are found in the octameric pore, corresponding to E106 and K145 of SEQ ID NO.: 1 and K142 and D106 of SEQ ID NO.: 4.

The modified leukocidin components of the invention are Staphylococcal antigens, suitably S. aureus antigens.

Because leukocidins are toxins, they need to be detoxified (i.e. rendered less-toxic or non-toxic to a mammal, e.g. human, when provided at a dosage suitable for protection) before they can be administered in vivo. The modified leukocidin components of the invention and complexes thereof are detoxified relative to unmutated, full-length leukocidins components and complexes thereof, such that the ability of a complex comprising at least one of the of the leukocidins of the invention to induce killing of target cells is decreased compared a complex comprising unmodified leukocidins.

A leukocidin component of the invention may be or are genetically detoxified. The genetically detoxified sequences may remove undesirable activities such as the ability to form a lipid-bilayer penetrating pore, membrane permeation, cell lysis, and cytolytic activity against suitable host target cells, in order to reduce toxicity, whilst retaining the ability to induce anti-leukocidin protective and/or neutralising antibodies following administration to a host (e.g. a human). For example, as described herein, a leukocidin component antigen may be altered so that it is unable to form a lipid-bilayer penetrating pore, whilst still maintaining its immunogenic epitopes.

Thus detoxication should not remove the antigen's ability to elicit antibodies that recognise epitopes of a polypeptide consisting of the amino acid sequence of SEQ ID NO.: 1 or SEQ ID NO.: 4 which are maintained in the sequence of the modified leucocidin component polypeptide, and/or of complexes thereof.

Host target cells are cells which can be affected by leukocidin-mediated cytotoxicity, which may be due to the expression of putative receptors for a given leukocidin (e.g. CCR5, CXCR1, and/or CXCR2 for LukED).

Suitable assays to assess detoxification of modified leukocidin components and complexes thereof relative to unmutated leukocidin components and complexes thereof include in vitro cytotoxicity assays. Methods of performing in vitro cytotoxicity assays are well known by those skilled in the art. Preferred cytotoxicity assays are performed by adding to THP-1 cell line (ATCC: TIB-202) serial dilutions of leukocidin (i.e., complexes comprising leucocidin components). Complexes may be generated by mixing each single component at a concentration of 10-50 μM (e.g. 20 μM) and incubation for 30 minutes at room temperature. Cells can be generally incubated with the leukocidin complex for 1 hour at 37° C., 5% CO₂, followed by the addition of a reagent suitable for measuring cell proliferation, such as CellTiter (Promega) and incubation for an additional 1-6 hours (e.g. 4 hours) at 37° C., 5% CO₂. Cell proliferation may be evaluated by measuring the signal emitted by the reagent used.

For example, a suitable modified leukocidin component of the invention may be one whose complexes exhibits lower cytotoxicity compared to complexes comprising only unmodified leukocidin components (e.g. measured via an in vitro cytotoxicity assay). For instance, the viability of target cells, as measured by a suitable in vitro cytotoxicity assay, after treatment with a leukocidin comprising a suitable modified leukocidin component may be higher, such as greater or equal to 1.5-fold, greater or equal to 2-fold, greater or equal to 3-fold, greater or equal to 4-fold, greater or equal to 10-fold, greater or equal to 20-fold, greater or equal to 50-fold, greater or equal to 100-fold, greater or equal to 1000-fold compared to the viability of target cells after treatment with a leukocidin comprising only unmodified leukocidin components, such as a polypeptide consisting of the amino acid sequence of SEQ ID NO.: 1 or SEQ ID NO.: 4. Assays may be used as described above and/or in the Examples.

Detoxification can be achieved by deleting a portion of LukE and/or LukD amino acid sequences. Preferably, the deletion affects the ability of LukE and/or LukD to form a lipid-bilayer penetrating pore. Suitably, the portion of LukE and/or LukD amino acid sequences which is deleted can be replaced by a heterologous amino acid sequence. The heterologous sequence may be a linker.

Polypeptides

Thus the polypeptide of the invention is a modified Staphylococcal leukocidin component polypeptide in which the stem domain has been fully or partially deleted, wherein the unmodified leukocidin component polypeptide comprises or consists of an amino acid sequence having at least 80%, 85%, 90%, 92%, 95%, 96%, 97%, 98%, 99%, or 100% sequence identity to a polypeptide comprising the amino acid sequence of SEQ ID NO.: 1 or a polypeptide comprising the amino acid sequence of SEQ ID NO.: 4. In one embodiment, the stem domain is fully or at least partially deleted respective to an unmodified leukocidin component polypeptide.

The structural features of LukE, LukD and LukED are described in detail in Nocadello S et al., Acta Crystallogr D Struct Biol. 2016 January; 72(Pt 1):113-20 and the stem region is defined accordingly. The amino acid numbers referred to herein correspond to the amino acids in of SEQ ID NO.: 1 or of SEQ ID NO.: 4 and a person skilled in the art can determine equivalent amino acid positions in an amino acid sequence at least 80%, 85%, 90%, 92%, 95%, 96%, 97%, 98% or 99% identical to SEQ ID NO.: 1 or SEQ ID NO.: 4 by alignment.

Thus the unmodified stem domain of the unmodified leukocidin component polypeptide may comprise or consist of amino acids E107 to Q146 of SEQ ID NO.: 1, or amino acids T104 to K142 of SEQ ID NO.: 4.

Said deletion within the stem domain is meant to encompass all or parts of the stem domain and, optionally, all or parts of the cap and/or rim domains.

In one embodiment the deletion is located between amino acids G79 and H169 of SEQ ID NO.: 1, such as between amino acids Y82 and A168, E89 and K160, A93 and K156, P101 and Y149, E106 and Y149, E106 and S148, E106 and E147, E106 and Q146, E106 and K145, E106 and S137, E106 and L132, E106 and Y118, E106 and Y116, E107 and Y149, E107 and S148, E107 and E147, E107 and Q146, E107 and K145, E107 and S137, E107 and L132, E107 and Y118, E107 and Y116, Y116 and L132, Y116 and S137, Y116 and K145, L132 and S137, L132 and K145, S137 and K145 of SEQ ID NO.: 1. In a preferred embodiment, the deletion is located between amino acids E106 and Y149 of SEQ ID NO.: 1. In a further preferred embodiment, the deletion is located between amino acids E106 and K145 of SEQ ID NO.: 1. In a particularly preferred embodiment, the deletion is located between amino acids E107 and Q146 of SEQ ID NO.: 1.

In one embodiment the deletion is located between amino acids P77 and N163 of SEQ ID NO.: 4, such as between amino acids Y80 and A162, K87 and K154, N90 and K150, P98 and Y144, T104 and Y144, T104 and S143, T104 and K142, T104 and Q141, T104 and T140, T104 and N132, T104 and P123, T104 and 1115, T104 and Y113, D106 and Y144, D106 and S143, D106 and K142, D106 and Q141, D106 and T140, D106 and N132, D106 and P123, D106 and 1115, D106 and Y113, Y113 and P123, Y113 and N132, Y113 and T140, P123 and N132, P123 and T140, N132 and T140 of SEQ ID NO.: 4. In a preferred embodiment, the deletion is located between amino acids T104 and K142 of SEQ ID NO.: 4.

In a particularly preferred embodiment, the deletion is located between amino acids D106 and K142 of SEQ ID NO.: 4.

In one embodiment, at least 5, 10, 15, 20, 30, 40, 50 60, 70 or 90 amino acids of the unmodified leukocidin component polypeptide are deleted. In one embodiment, the at least 5 amino acid which are deleted are consecutive. In a preferred embodiment, about 40 consecutive amino acids of a the unmodified leukocidin component polypeptide are deleted. In a preferred embodiment, where the unmodified leukocidin component polypeptide is SEQ ID NO.: 1, residue Y116 of SEQ ID NO.: 1 is deleted, and where the unmodified leukocidin component polypeptide is SEQ ID NO.: 4, residue Y113 of SEQ ID NO.: 4 is deleted.

In one embodiment, additional 1 to 10 amino acids flanking the N-terminus and/or at the C-terminus of the stem domain of an unmodified leukocidin component polypeptide may be deleted, for example, 1, 2, 3, 4, 5, 6, 7, 8, 9 or 10 amino acids flanking the N-terminus and/or at the C-terminus of the stem domain may be deleted.

In one embodiment, the polypeptide of the invention may further comprise 1, 2, 3, 4, or 5 single amino acid substitutions, deletions and/or insertions.

The invention also provides a modified leukocidin polypeptide comprising an amino acid sequence having at least 80%, 85%, 90%, 92%, 95%, 96%, 97%, 98%, 99%, or 100% sequence identity to a polypeptide comprising an N-terminal and/or C-terminal fragment of SEQ ID NO.: 1 or SEQ ID NO.: 4. In one embodiment, the polypeptide optionally comprises 1, 2, 3, 4, or 5 single amino acid substitutions, deletions and/or insertions.

In one embodiment, the N-terminal fragment of SEQ ID NO.: 1 comprises amino acids 1-79, 1-82, 1-89, 1-93, 1-101, 1-106, 1-107, 1-115, 1-118, or 1-132 of SEQ ID NO.: 1. In a preferred embodiment, the N-terminal fragment of SEQ ID NO.: 1 comprises amino acids 1-106 of SEQ ID NO.:

In one embodiment, the C-terminal fragment of SEQ ID NO.: 1 comprises amino acids 116-301, 132-301, 137-301, 145-301, 146-301, 147-301, 148-301, 149-301, 156-301, 160-301, 168-301, or 169-301 of SEQ ID NO.: 1. In a preferred embodiment, the C-terminal fragment of SEQ ID NO.: 1 comprises amino acids 147-301 of SEQ ID NO.: 1.

In one embodiment, the N-terminal fragment of SEQ ID NO.: 4 comprises amino acids 1-77, 1-80, 1-87, 1-90, 1-98, 1-103, 1-104, 1-113, 1-115, or 1-123 of SEQ ID NO.: 4. In a preferred embodiment, the N-terminal fragment of SEQ ID NO.: 4 comprises amino acids 1-103 of SEQ ID NO.: 4.

In one embodiment, the C-terminal fragment of SEQ ID NO.: 4 comprises amino acids 113-283, 115-283, 123-283, 132-283, 123-283, 140-283, 141-283, 142-283, 143-283, 144-283, 150-283, 154-283, 162-283, or 163-283 of SEQ ID NO.: 4. In a preferred embodiment, the C-terminal fragment of SEQ ID NO.: 4 comprises amino acids 143-283 of SEQ ID NO.: 4.

In one embodiment, the modified leukocidin component may comprise or consist of said N-terminal fragment of SEQ ID NO.: 1 or SEQ ID NO.: 4 and the C-terminal fragment of a heterologous leukocidin. In one embodiment, the modified leukocidin component may comprise or consist of the N-terminal fragment of a heterologous leukocidin component and said C-terminal fragment SEQ ID NO.: 1 or SEQ ID NO.: 4. When said leucocidin component is SEQ ID NO.: 1, a heterologous leukocidin component may be or is a leukocidin component different from SEQ ID NO.:1 (LukD), such as LukF-PV. When said leucocidin component is SEQ ID NO.: 1, a heterologous leukocidin component may be or is a leukocidin component different from SEQ ID NO.:1 (LukE), such as LukS-PV.

Thus leukocidin components of the invention may comprise an amino acid sequence having at least 80%, 85%, 90%, 92%, 95%, 96%, 97%, 98%, 99%, or 100% sequence identity to SEQ ID NO.: 2 or SEQ ID NO.: 5. In one embodiment, the polypeptide of the invention may further comprise 1, 2, 3, 4, or 5 single amino acid substitutions, deletions and/or insertions.

In one aspect, the amino acid sequence which is deleted within the stem domain of the said leukocidin component can replaced by a linker. Suitably, the stem region of said leukocidin component or a fragment thereof is deleted and can be replaced by a linker. Linker are suitably peptides that are used for connection of at least one other peptide or protein. The linker may be a flexible polypeptide comprising at least 2 to 50 amino acids, such as 2, 3, 4, 5, 10, 20, 30, 40, 50 amino acids. The linker may suitably comprise alanine, serine and/or glycine residues. In a preferred embodiment, the linker comprises 4 amino acids. In a particularly preferred embodiment, the linker comprises a sequence of SEQ ID NO.: 7.

Thus the leukocidin components of the invention may comprise an amino acid sequence having at least 80%, 85%, 90%, 92%, 95%, 96%, 97%, 98%, 99%, or 100% sequence identity to SEQ ID NO.: 3 or SEQ ID NO.: 6. The polypeptide of the invention may further comprise 1, 2, 3, 4, or 5 single amino acid substitutions, deletions and/or insertions.

In an embodiment, the leukocidin component of the invention further comprises a “peptide tag” or “tag”, i.e. a sequence of amino acids that allows for the isolation and/or identification of the modified leukocidin component. For example, adding a tag to a modified leukocidin component of the invention can be useful in the purification of that antigen. Exemplary tags that can be used herein include, without limitation, histidine (HIS) tags. In one embodiment, the tag is a hexa-histidine tag. In another embodiment, the tag is a HR tag, for example an HRHR tag (SEQ ID NO: 11). In certain embodiments, the tags used herein are removable, e.g. removal by chemical agents or by enzymatic means, once they are no longer needed, e.g. after the antigen has been purified. Optionally the peptide tag is located at the N-terminus of the amino acid sequence. Optionally the peptide tag comprises six histidine residues at the N-terminus of the amino acid sequence. Optionally the peptide tag comprises four HR residues (HRHR) at the N-terminus of the amino acid sequence. The peptide tag may comprise or be preceded by one, two or more additional amino acid residues, for example alanine, serine and/or glycine residues, e.g. GS.

Nucleic Acids

A further aspect of the invention provides a polynucleotide encoding the polypeptide of the invention as defined above.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acids of the invention can take various forms e.g. single-stranded, double-stranded, vectors, primers, probes, labelled, unlabelled, etc.

Nucleic acids of the invention are preferably in isolated or substantially isolated form.

The term “nucleic acid” includes DNA and RNA, and also their analogues, such as those containing modified backbones, and also peptide nucleic acids (PNA), etc.

The invention also provides vectors (such as plasmids) comprising nucleotide sequences of the invention (e.g. cloning or expression vectors, such as those suitable for nucleic acid immunisation) and host cells transformed with such vectors.

Host Cells

Also provided is a host cell comprising a vector or polynucleotide of the invention. The host cell may be a bacterium. The bacterium may be E. coli. The bacterium may be transfected with the vector or the polynucleotide of the invention and may constitutively express the polypeptide, but in some embodiments expression may be under the control of an inducible promoter. The bacterium may hyper-express the polypeptide.

Complexes

The modified leukocidin components of the invention can be protomers (i.e. subunits) of an oligomeric complex. Said complex comprising a modified leukocidin component of the invention may be unable to form a pore-forming toxin and is thus detoxified.

Complexes of the invention may comprise mutated leukocidin components of the invention. Suitably, complexes of the invention may comprise mutated LukE and/or LukD polypeptides of the invention as defined above in equimolar ratio, such as 4 LukE components and 4 LukD components. Suitably, all the LukE protomers within a complex may be identical and all the LukD protomers within a complex may be identical.

In one embodiment, a complex of the invention may comprise a modified LukE and/or a modified LukD polypeptide component as defined above. Suitably, the complex may be detoxified.

In one embodiment, the invention provides a complex comprising a polypeptide comprising an amino acid sequence an amino acid sequence having at least 80%, 85%, 90%, 92%, 95%, 96%, 97%, 98%, 99%, or 100% sequence identity to SEQ ID NO.: 3, and/or a polypeptide comprising an amino acid sequence having at least 80%, 85%, 90%, 92%, 95%, 96%, 97%, 98%, 99%, or 100% sequence identity to SEQ ID NO.: 5.

In one embodiment, the invention provides a complex comprising a polypeptide comprising an amino acid sequence an amino acid sequence having at least 80%, 85%, 90%, 92%, 95%, 96%, 97%, 98%, 99%, or 100% sequence identity to SEQ ID NO.: 3, further comprising 1, 2, 3, 4, or 5 single amino acid substitutions, deletions and/or insertions and/or a polypeptide comprising an amino acid sequence having at least 80%, 85%, 90%, 92%, 95%, 96%, 97%, 98%, 99%, or 100% sequence identity to SEQ ID NO.: 5, further comprising 1, 2, 3, 4, or 5 single amino acid substitutions, deletions and/or insertions.

Immunogenic Compositions and Vaccines

In one aspect, it is provided an immunogenic composition comprising the polypeptide or the complex of the invention as described above.

In one embodiment, the immunogenic composition of the invention comprises additional antigens. In a preferred embodiment, the immunogenic composition comprises a ClfA antigen, a Hla antigen, a SpA antigen, and a staphylococcal capsular polysaccharide.

Clumping factor A (ClfA) is an important S. aureus adhesin which is required for virulence and helps the bacteria evade host defence mechanisms. It binds to fibrinogen in the ECM, aiding in adherence and colonisation of host tissues and additionally causing cell clumping and coating of the bacterial cells in fibrinogen, which promotes immune evasion by impairing deposition of opsonins on the bacteria.

ClfA is present in nearly all S. aureus strains. It is an important virulence factor, contributing to the pathogenesis of septic arthritis and endocarditis. ClfA binds to the C-terminus of the γ-chain of fibrinogen and is thereby able to induce clumping of bacteria in fibrinogen solution. Expression of ClfA on S. aureus hampers phagocytosis by both macrophages and neutrophils. In neutrophils this is due to both a fibrinogen-dependent and to a fibrinogen-independent mechanism. In contrast, platelets are activated by bacteria expressing ClfA through its interaction with GPIIb/IIIa leading to aggregation. This is most efficiently executed when fibrinogen is present, but there is also a fibrinogen-independent pathway for platelet activation.

Suitable ClfA antigens used with the invention are described in WO2019/158537.

In a preferred embodiment, the ClfA antigen comprises an amino acid sequence having at least 80%, 85%, 90%, 92%, 95%, 96%, 97%, 98%, 99%, or 100% sequence identity to SEQ ID NO: 8. In a particularly preferred embodiment, the ClfA antigen comprises the amino acid sequence of SEQ ID NO: 8.

Hla is an important secreted staphylococcal toxin. It creates a lipid-bilayer penetrating pore in the membrane of human erythrocytes and other cells, resulting in cell lysis.

Suitable Hla antigens used with the invention are described in WO2019/158537.

In a preferred embodiment, the Hla antigen comprises the amino acid sequence having at least 80%, 85%, 90%, 92%, 95%, 96%, 97%, 98%, 99%, or 100% sequence identity to SEQ ID NO: 9. In a particularly preferred embodiment, the Hla antigen comprises the amino acid sequence of SEQ ID NO: 9.

The wild-type SpA (staphylococcal protein A) is a cell wall-anchored surface protein which is a crucial virulence factor for lung infections, septicaemia, and abscess development and is expressed by most clinical S. aureus isolates. Wild-type SpA binds to the Fc portion of human IgG, to V_{H}3-containing B cell receptors, to von Willebrand factor at its A1 domain, and to the TNF-α receptor 1. Interaction of SpA with B cell receptors affects B cell development with effects on adaptive and innate immune responses, whereas its binding to the Fcγ of IgG interferes with opsonophagocytic clearance of staphylococci by polymorphonuclear leukocytes. The N-terminal part of mature SpA is comprised of four or five 56-61-residue Ig-binding domains, which fold into triple helical bundles connected by short linkers, and are designated in order E, D, A, B, and C. These domains display ˜80% identity at the amino acid level, are 56 to 61 residues in length, and are organized as tandem repeats. The C-terminal region is comprised of “Xr”, a highly repetitive yet variable octapeptide, and “Xc”, a domain which abuts the cell wall anchor structure of SpA.

Suitable SpA antigens used with the invention are described in WO2019/158537.

In one embodiment, the SpA antigen comprises an amino acid sequence having at least 80%, 85%, 90%, 92%, 95%, 96%, 97%, 98%, 99%, or 100% sequence identity to of SEQ ID NO: 10; in an alternative embodiment, the SpA antigen may comprise a fragment of at least ‘n’ consecutive amino acids of SEQ ID NO: 10, wherein ‘n’ is 7 or more (e.g. 8, 10, 12, 14, 16, 18, 20, 25, 30, 35, 40, 50, 60, 70, 80, 90, 100, 150, 200, 250 or more).

The compositions of the invention may further comprise a bacterial capsular saccharide. The bacterial capsular saccharide may be selected from a S. aureus serotype 5 or 8 capsular saccharide. 75% of S. aureus strains express either Type 5 (CP5) or Type 8 (CP8) capsular polysaccharide, so a vaccine comprising CP5 and CP8 could potentially provide protection against the majority of circulating S. aureus strains.

Suitable polysaccharide antigens used with the invention are described in WO2019/158537.

In one embodiment, the capsular polysaccharide may be conjugated to a carrier protein. In one embodiment, the capsular polysaccharide may a S. aureus serotype 5 and/or type 8 capsular polysaccharide. In one embodiment, the capsular polysaccharide may be conjugated to one of ClfA antigen, a Hla antigen, a SpA antigen. In a preferred embodiment, the immunogenic composition may comprise a S. aureus serotype 5 capsular polysaccharide conjugated to a Hla antigen and/or a type 8 capsular polysaccharide conjugated to a ClfA antigen. In a preferred embodiment, said ClfA-type 8 capsular polysaccharide and Hla-serotype 5 capsular polysaccharide conjugates are bioconjugates. Suitable antigens and bioconjugates used with the invention are described in WO2019/158537.

The immunogenic composition of the invention may be formulated into a pharmaceutical composition. According to one aspect, the invention provides a pharmaceutical composition comprising an immunogenic composition of the invention and a pharmaceutically acceptable excipient or carrier.

In one aspect, it is provided a vaccine comprising the immunogenic composition of the invention. The present invention also provides a vaccine comprising an immunogenic composition of the invention and a pharmaceutically acceptable excipient or carrier.

Pharmaceutically acceptable excipients and carriers can be selected by those of skill in the art. Numerous pharmaceutically acceptable excipients and carriers are described, for example, in Remington's Pharmaceutical Sciences, by E. W. Martin, Mack Publishing Co. Easton, Pa., 5th Edition (975).

In an embodiment, the compositions of the invention have a pharmaceutically acceptable osmolality to avoid cell distortion or lysis. A pharmaceutically acceptable osmolality will generally mean that solutions will have an osmolality which is approximately isotonic or mildly hypertonic.

Immunogenic compositions comprise an immunologically effective amount of the protein or conjugate (e.g. bioconjugate) of the invention, as well as any other components. By “immunologically effective amount”, it is meant that the administration of that amount to an individual, either as a single dose or as part of a series is effective for treatment or prevention. This amount varies depending on the health and physical condition of the individual to be treated, age, the degree of protection desired, the formulation of the vaccine and other relevant factors. It is expected that the amount will fall in a relatively broad range that can be determined through routine trials.

Vaccine preparation is generally described in Vaccine Design (“The subunit and adjuvant approach” (eds Powell M. F. & Newman M. J.) (1995) Plenum Press New York).

In one embodiment, the immunogenic composition of the invention comprises an adjuvant. In an embodiment, the immunogenic compositions of the invention comprise, or are administered in combination with, an adjuvant.

Adjuvants can enhance an immune response by several mechanisms including, e.g. lymphocyte recruitment, stimulation of B and/or T cells, and stimulation of macrophages. In an embodiment, the adjuvant is selected to be a preferential inducer of either a TH1 or a TH2 type of response, preferably a TH1 type response. High levels of Th1-type cytokines tend to favour the induction of cell mediated immune responses to a given antigen, whilst high levels of Th2-type cytokines tend to favour the induction of humoral immune responses to the antigen.

Adjuvants which may be used in compositions of the invention include, but are not limited to insoluble metal salts, oil-in-water emulsions (e.g. MF59 or AS03, both containing squalene), saponins, non-toxic derivatives of LPS (such as monophosphoryl lipid A or 3-O-deacylated MPL), immunostimulatory oligonucleotides, detoxified bacterial ADP-ribosylating toxins, microparticles, liposomes, imidazoquinolones, or mixtures thereof. Other substances that act as immunostimulating agents are well-known to those of skills in the art.

In one embodiment, the immunogenic composition comprises an aluminium hydroxide and/or aluminium phosphate adjuvant, and polypeptides are generally adsorbed to these salts. These salts include oxyhydroxides and hydroxyphosphates. The salts can take any suitable form (e.g. gel, crystalline, amorphous, etc.).

In one embodiment the adjuvant comprises both a TLR4 agonist and immunologically active saponin. In an embodiment, the TLR4 agonist is a lipopolysaccharide. Suitably the saponin comprises an active fraction of the saponin derived from the bark of Quillaja Saponaria Molina, such as QS21. Suitably the lipopolysaccharide is a Lipid-A derivative such as 3D-MPL. In a specific embodiment, the lipopolysaccharide is 3D-MPL and the immunologically active saponin is QS21. In an embodiment, said adjuvant composition comprises a lipopolysaccharide and immunologically active saponin in a liposomal formulation. Suitably in one form of this embodiment, the adjuvant consists essentially of 3D-MPL and QS21, with optionally sterol which is preferably cholesterol.

In one embodiment, the adjuvant comprises a saponin and a lipopolysaccharide (LPS). In one embodiment, the adjuvant comprises a saponin, a lipopolysaccharide in a liposomal formation. In one embodiment, the saponin is an immunologically active saponin fraction derived from the bark of Quillaja Saponaria Molina. In a preferred embodiment, the saponin is QS21. In one embodiment, the lipopolysaccharide is a lipid A derivative. In a preferred embodiment, the lipopolysaccharide is 3D-MPL.

In one embodiment, the immunogenic composition further comprises a sterol. In one preferred embodiment, the sterol is cholesterol.

Liposome size may vary from 30 nm to several um depending on the phospholipid composition and the method used for their preparation. In particular embodiments of the invention, the liposome size will be in the range of 50 nm to 500 nm and in further embodiments 50 nm to 200 nm. Optimally, the liposomes should be stable and have a diameter of ˜100 nm to allow sterilisation by filtration.

Other TLR4 agonists which may be of use in the present invention include Glucopyranosyl Lipid Adjuvant (GLA) such as described in WO2008/153541 or WO2009/143457 or the literature articles Coler R N et al. (2011) Development and Characterization of Synthetic Glucopyranosyl Lipid Adjuvant System as a Vaccine Adjuvant. PLoS ONE 6(1): e16333. doi:10.1371/journal.pone.0016333 and Arias M A et al. (2012) Glucopyranosyl Lipid Adjuvant (GLA), a Synthetic TLR4 Agonist, Promotes Potent Systemic and Mucosal Responses to Intranasal Immunization with HIVgp140. PLoS ONE 7(7): e41144. doi:10.1371/journal.pone.0041144. WO2008/153541 or WO2009/143457 are incorporated herein by reference for the purpose of defining TLR4 agonists which may be of use in the present invention. 4′-monophosporyl lipid A (MPL), which may be obtained by the acid hydrolysis of LPS extracted from a deep rough mutant strain of gram-negative bacteria, retains the adjuvant properties of LPS while demonstrating a toxicity which is reduced by a factor of more than 1000 (as measured by lethal dose in chick embryo eggs) (Johnson et al. 1987 Rev. Infect. Dis. 9 Suppl:S512-S516). LPS is typically refluxed in mineral acid solutions of moderate strength (e.g. 0.1 M HCl) for a period of approximately 30 minutes. This process results in dephosphorylation at the 1 position, and decarbohydration at the 6′ position, yielding MPL.

3-O-deacylated monophosphoryl lipid A (3D-MPL), which may be obtained by mild alkaline hydrolysis of MPL, has a further reduced toxicity while again maintaining adjuvanticity, see U.S. Pat. No. 4,912,094 (Ribi Immunochemicals). Alkaline hydrolysis is typically performed in organic solvent, such as a mixture of chloroform/methanol, by saturation with an aqueous solution of weak base, such as 0.5 M sodium carbonate at pH 10.5. Further information on the preparation of 3D-MPL is available in, for example, U.S. Pat. No. 4,912,094 and WO02/078637 (Corixa Corporation).

Quillaja saponins are a mixture of triterpene glycosides extracted from the bark of the tree Quillaja saponaria. Crude saponins have been extensively employed as veterinary adjuvants. Quil-A is a partially purified aqueous extract of the Quillaja saponin material. Quil-A is a saponin preparation isolated from the South American tree Quillaja Saponaria Molina and was first described by Dalsgaard et al. in 1974 (“Saponin adjuvants”, Archiv. für die gesamte Virusforschung, Vol. 44, Springer Verlag, Berlin, p 243-254) to have adjuvant activity. Purified fragments of Quil A have been isolated by HPLC which retain adjuvant activity without the toxicity associated with Quil A (EP 0 362 278), for example QS7 and QS21 (also known as QA7 and QA21). QS-21 is a natural saponin derived from the bark of Quillaja saponaria Molina, which induces CD8+ cytotoxic T cells (CTLs), Th1 cells and a predominant IgG2a antibody response and is a preferred saponin in the context of the present invention. QS21 is a HPLC purified non-toxic fraction of Quil A and its method of production is disclosed (as QA21) in U.S. Pat. No. 5,057,540. Preferably the adjuvant contains QS21 in substantially pure form, that is to say, the QS21 is at least 90% pure, for example at least 95% pure, or at least 98% pure.

Adjuvants containing combinations of lipopolysaccharide and Quillaja saponins have been disclosed previously, for example in EP0671948. This patent demonstrated a strong synergy when a lipopolysaccharide (3D-MPL) was combined with a Quillaja saponin (QS21). Good adjuvant properties may be achieved with combinations of lipopolysaccharide and quillaja saponin as immunostimulants in an adjuvant composition even when the immunostimulants are present at low amounts in a human dose, as described in WO2007/068907.

In a specific embodiment, QS21 is provided in its less reactogenic composition where it is quenched with an exogenous sterol, such as cholesterol for example. Several particular forms of less reactogenic compositions wherein QS21 is quenched with an exogenous cholesterol exist. In a specific embodiment, the saponin/sterol is in the form of a liposome structure (WO 96/33739, Example 1). In this embodiment the liposomes suitably contain a neutral lipid, for example phosphatidylcholine, which is suitably non-crystalline at room temperature, for example egg yolk phosphatidylcholine, dioleoyl phosphatidylcholine (DOPC) or dilauryl phosphatidylcholine. The liposomes may also contain a limited amount of a charged lipid which increases the stability of the liposome-saponin structure for liposomes composed of saturated lipids. In these cases, the amount of charged lipid is suitably 1-20% w/w, preferably 5-10% w/w of the liposome composition. Suitable examples of such charged lipids include phosphatidylglycerol and phosphatidylserine. Suitably, the neutral liposomes will contain less than 5% w/w charged lipid, such as less than 3% w/w or less than 1% w/w. The ratio of sterol to phospholipid is 1-50% (mol/mol), suitably 20-25%.

Suitable sterols include β-sitosterol, stigmasterol, ergosterol, ergocalciferol and cholesterol. In one particular embodiment, the adjuvant composition comprises cholesterol as sterol. These sterols are well known in the art, for example cholesterol is disclosed in the Merck Index, 11th Edn., page 341, as a naturally occurring sterol found in animal fat.

Where the active saponin fraction is QS21, the ratio of QS21: sterol will typically be in the order of 1:100 to 1:1 (w/w), suitably between 1:10 to 1:1 (w/w), and preferably 1:5 to 1:1 (w/w). Suitably excess sterol is present, the ratio of QS21:sterol being at least 1:2 (w/w). In one embodiment, the ratio of QS21:sterol is 1:5 (w/w). The sterol is suitably cholesterol.

3D-MPL is sold under the name MPL by GlaxoSmithKline Biologicals S.A. and is referred throughout the document as MPL or 3D-MPL. see, for example, U.S. Pat. Nos. 4,436,727; 4,877,611; 4,866,034 and 4,912,094. 3D-MPL primarily promotes CD4+ T cell responses with an IFN-γ (Th1) phenotype. 3D-MPL can be produced according to the methods disclosed in GB 2 220 211 A. Chemically it is a mixture of 3-deacylated monophosphoryl lipid A with 3, 4, 5 or 6 acylated chains. Preferably in the compositions of the present invention small particle 3D-MPL is used. Small particle 3D-MPL has a particle size such that it may be sterile-filtered through a 0.22 μm filter. Such preparations are described in WO 94/21292.

The adjuvant AS01 comprises 3D-MPL and QS21 in a quenched form with cholesterol, and was made as described in WO 96/33739. In particular the AS01 adjuvant was prepared essentially as Example 1.1 of WO 96/33739. The AS01_{B} adjuvant comprises: liposomes, which in turn comprise dioleoyl phosphatidylcholine (DOPC), cholesterol and 3D MPL [in an amount of 1000 μg DOPC, 250 μg cholesterol and 50 μg 3D-MPL, each value given approximately per vaccine dose], QS21 [50 μg/dose], phosphate NaCl buffer and water to a volume of 0.5 ml.

The AS01_{E} adjuvant comprises the same ingredients as AS01_{B} but at a lower concentration in an amount of 500 μg DOPC, 125 μg cholesterol, 25 μg 3D-MPL and 25 μg QS21, phosphate NaCl buffer and water to a volume of 0.5 ml.

In a preferred embodiment, the adjuvant used in the present invention is AS01_{E}.

Method of Administration

Compositions of the invention will generally be administered directly to a patient. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. In one aspect, the immunogenic composition or vaccine of the invention is administered by the intramuscular delivery route. Intramuscular administration may be to the thigh or the upper arm. Injection is typically via a needle (e.g. a hypodermic needle). A typical intramuscular dose is 0.5 ml.

Dosage treatment can be a single dose schedule or a multiple dose schedule. Multiple doses may be used in a primary immunisation schedule and/or in a booster immunisation schedule. A primary dose schedule may be followed by a booster dose schedule. Suitable timing between priming doses, and between priming and boosting, can be routinely determined.

Prophylactic and Therapeutic Uses

The present invention provides methods of treating and/or preventing bacterial infections of a subject comprising administering to the subject a polypeptide of the invention, a complex of the invention, an immunogenic composition of the invention, or a vaccine of the invention. In a specific embodiment, the polypeptide of the invention, the complex of the invention, the immunogenic composition of the invention, or the vaccine of the invention is used in the prevention of infection of a subject (e.g. human subjects) by a staphylococcal bacterium. S. aureus infects various mammals (including cows, dogs, horses, and pigs), but the preferred subject for use with the invention is a human.

In a specific embodiment, the polypeptide of the invention, the complex of the invention, the immunogenic composition of the invention, or the vaccine of the invention is used to treat or prevent an infection by Staphylococcus species, in particular S. aureus. For example, the polypeptide of the invention, the complex of the invention, the immunogenic composition of the invention, or the vaccine of the invention may be used to prevent against S. aureus infection, including a nosocomial infection.

Also provided are methods of inducing an immune response in a subject against a staphylococcal bacterium, in particular S. aureus, comprising administering to the subject a polypeptide of the invention, a complex of the invention, an immunogenic composition of the invention, or a vaccine of the invention. In one embodiment, said subject has bacterial infection at the time of administration. In another embodiment, said subject does not have a bacterial infection at the time of administration. The polypeptide of the invention, the complex of the invention, the immunogenic composition of the invention, or the vaccine of the invention can be used to induce an immune response against Staphylococcus species, in particular S. aureus.

Also provided are methods of inducing the production of opsonophagocytic antibodies in a subject against a staphylococcal bacterium, in particular S. aureus, comprising administering to the subject a polypeptide of the invention, a complex of the invention, an immunogenic composition of the invention, or a vaccine of the invention. In one embodiment, said subject has bacterial infection at the time of administration. In another embodiment, said subject does not have a bacterial infection at the time of administration.

Also provided are methods of inducing the production of antibodies able to neutralise or reduce the activity of staphylococcal Leukocidins in a subject, comprising administering to the subject a polypeptide of the invention, a complex of the invention, an immunogenic composition of the invention, or a vaccine of the invention. Said Leukocidin activity may be ability to lyse human immune cells.

Also provided is a method of immunising a human host against staphylococcal infection, particularly S. aureus infection, comprising administering to the host a polypeptide of the invention, a complex of the invention, an immunogenic composition of the invention, or a vaccine of the invention.

Also provided is a method of inducing an immune response to a staphylococcal bacterium, in particular S. aureus, in a subject, the method comprising administering a therapeutically or prophylactically effective amount of a polypeptide of the invention, a complex of the invention, an immunogenic composition of the invention, or a vaccine of the invention.

Also provided is a polypeptide of the invention, a complex of the invention, an immunogenic composition of the invention, or a vaccine of the invention for use in a method of treatment and/or prevention of disease, for example for use a method of treatment or prevention of a disease caused by staphylococcal infection, particularly S. aureus infection.

Also provided is a polypeptide of the invention, a complex of the invention, an immunogenic composition of the invention, or a vaccine of the invention in the manufacture of a medicament for the treatment or prevention of a disease caused by staphylococcal infection, particularly S. aureus infection.

Also provided is the use of a polypeptide of the invention, a complex of the invention, an immunogenic composition of the invention, or a vaccine of the invention for the manufacture of a medicament for use in a method of treatment and/or prevention of disease, for example for use a method of treatment or prevention of a disease caused by staphylococcal infection, particularly S. aureus infection.

Also provided is a pharmaceutical for treatment or prevention of staphylococcal infection, particularly S. aureus infection, comprising a polypeptide of the invention, a complex of the invention, an immunogenic composition of the invention, or a vaccine of the invention.

Also provided is a polypeptide of the invention, a complex of the invention, an immunogenic composition of the invention, or a vaccine of the invention for use in a method of inducing an immune response in a subject against a staphylococcal bacterium, in particular S. aureus.

Also provided is a polypeptide of the invention, a complex of the invention, an immunogenic composition of the invention, or a vaccine of the invention for use in a method of inducing the production of opsonophagocytic antibodies in a subject against a staphylococcal bacterium, in particular S. aureus.

Also provided is a polypeptide of the invention, a complex of the invention, an immunogenic composition of the invention, or a vaccine of the invention for use in a method of inducing the production of antibodies able to neutralise or reduce the activity of staphylococcal leukocidins in a subject, comprising administering to the subject a polypeptide of the invention, a complex of the invention, an immunogenic composition of the invention, or a vaccine of the invention. Said leukocidin activity may be ability to lyse human immune cells.

All references or patent applications cited within this patent specification are incorporated by reference herein.

In order that this invention may be better understood, the following examples are set forth. These examples are for purposes of illustration only, and are not to be construed as limiting the scope of the invention in any manner.

### EXAMPLES

### Example 1: Bacterial Strains and Culture Conditions

S. aureus strains used in this study are listed in Table 1. Strains were grown at 37° C. in Tryptic soy broth (TSB, Difco Laboratories) or in trypticase soy agar (TSA) supplemented with 10 μg ml 1 of chloramphenicol and 5% (v/v) of sheep blood if required. For preparation of bacterial challenge inoculum for infection studies in animals, an aliquot of bacterial (2 ml) frozen in PBS (Phosphate-buffered saline)+Bovine serum albumin (BSA) 10% (w/v)+glutamate 10% (w/v) was thawed, inoculated in 48 ml of TSB (starting from and optical density of 0.05) in flasks and incubated at 37° C. at 250 rpm until the optical density at 600 nm reached 2. Bacteria were washed twice in equal volumes of PBS, collected by centrifugation for 10 minutes at 4000 rpm and suspended to 10⁸ CFU ml⁻¹ to reach the necessary concentration for infection (10⁷ CFU per infectious dose).

TABLE 1
Strains and plasmids

| Strains or plasmid | Relevant characteristics |
|---|---|
| S. aureus strains | |
| Newman | MSSA, clumping factor overproducer, UK, CC8, |
| | CPS 5, GenBank accession number NC_009641 |
| USA300-(FPR3757 | GenBank accession number CP000255.1 |
| or LAC) | |
| USA100 Mu50 | GenBank accession number BA000017.4 |
| USA400 MW2 | GenBank accession number BA000033.2 |
| USA100 N315 | GenBank accession number BA000018.3 |
| COL | GenBank accession number CP000046.1 |

### Example 2: In Vitro and In Vivo Expression of LukE and LukD

Sample Collection, RNA Extraction and cDNA Synthesis.

S. aureus strain Newman was grown in TSB broth to early-, mid- and late-exponential, pre-stationary and stationary phase, and prepared mRNA for each timepoint. Bacterial mRNA samples for in vitro expression profiling were extracted from cells from TSA broth cultures grown to the indicated OD, and stabilised by RNAprotect Bacteria Reagent (QIAgen) according to the manufacturer's instructions.

Bacteria were thereafter collected by centrifugation for 10 min at 3200×g and 4° C. Bacterial pellets were then either directly processed for RNA extraction or stored at −80° C.

For samples from in vivo expression profiling, eight to ten-weeks old female CD1 mice (pathogen free) were infected intravenously with a sub-lethal dose of S. aureus (— 1×10⁷ CFU per mouse) and organs harvested at defined time points. Samples for RNA extraction were stabilized using RNAprotect Bacteria Reagent (QIAgen, Germany) according to the manufacturer's instructions. In case of extracted host organs, the organs were collected in gentleMACS M tubes (Milteny Biotech) containing 2 to 4 ml of RNAprotect and immediately homogenized. Larger cell debris was removed from the homogenized samples by centrifugation at 100×g for 5 minutes and bacteria were thereafter collected by centrifugation for 10 minutes at 3200×g. Bacterial pellets were then either directly processed for RNA extraction or stored at −80° C. For RNA extraction, the bacterial pellet from either in vitro cultures or in vivo infections was resuspended in 1 ml of Trizol reagent (Ambion) and lysed in a FastPrep®-24 homogenizer (MP Biomedicals) using three cycles of 60 s at 6.5 m s⁻² followed by 5 min incubation in ice after each cycle. RNA was extracted from the suspension using Direct-zol™ RNA MiniPrep Kit (Zymo Research) applying an on-column DNase digestion step using the RNase-free DNase kit (QIAgen) according to the manufacturer's instructions. Residual DNA was removed by a second DNase treatment using RQ1 DNase (Promega) followed by RNA purification using the PureLink kit (Ambion) according to the manufacturer's instructions. RNA quality was assessed by gel electrophoresis and Agilent 2100 Bioanalyzer and absence of contaminating DNA confirmed by qRT-PCR. cDNA was synthetized using the SuperScript First-Strand Synthesis System for RT-PCR (Invitrogen-Life Technologies) according to the manufacturer's instructions, using random hexamer primer for reverse transcription (RD on 300 to 4000 ng of total RNA.

Determination of Bacterial RNA Concentration in Host Extracted RNA Samples and qRT-PCR

Mouse RNA was spiked with defined quantities of bacterial RNA and cDNA prepared as described above. qRT-PCR was performed using Platinum SYBR Green qPCR SuperMix-UDG (Invitrogen-Life Technologies) using ROX as internal control on a STRATAGEN Mx3000P QPCR system using the following cycling parameters: 95° C. for 10 min; 45 cycles of 95° C. for 30 s, 55° C. for 30 s and 72° C. for 30 s; 95° C. for 1 min, 55° C. for 30 s and finally 95° C. for 30 s. The amount of bacterial RNA in each sample was determined using 16s rRNA primers Sa_16 s_+332_F SEQ ID NO: 12 (5′ GAGACACGGTCCAGACTCCT 3′) and Sa_16 s_+437_R SEQ ID NO: 13 (5′ ACGATCCGAAGACCTTCATC 3′) and relating each sample to a calibration curve.

Design of Taqman, Preamplification of cDNA Samples and Taqman qRT-PCR Assays.

Taqman qRT-PCR assays were designed on unique and conserved regions within the respective target genes of strains: Newman, USA300 FPR3757, Mu50, MW2, N315 and COL. Forward and reverse primers were designed to amplify fragments between 71 and 278 bp (Table 2). The quantities of cDNA prepared from 25 ng of RNA as described above were used for preamplification using TaqMan PreAmp Master Mix (Invitrogen) according to the manufacturer's instructions. In brief, assays to be tested were mixed to working concentration of 180 nM for each assay (0.2×) and used for preamplification as per manufacturer's instructions using the following cycling parameters: 95° C. for 10 min; 14 to 20 cycles (as indicated for each experiment) of 95° C. for 15 s and 60° C. for 4 min. Preamplified samples were diluted 1:5 in TE buffer (10 mM Tris, pH 8.0, 0.1 mM EDTA) prior to loading onto the Fluidigm 48.48 Dynamic Array™ IFC chip. qPCR were performed exactly as in the manufacturer's instructions using the following cycling parameters: 50° C. for 2 min, 95° C. for 10 min; 40 cycles of 95° C. for 15 s and 60° C. for 1 min. Samples were normalized to the expression levels of gyrB and relative expression values to the reference culture were calculated using the ΔΔct method.

TABLE 2

| | Forward | Reverse | |
|---|---|---|---|
| Target | Primer | Primer | Probe |
| gene | 5′-3′ | 5′-3′ | 5′-3′ |
| lukD | GAAAGTTA | ATTATTCA | CCCCAG |
| | CAGAACTA | TAATTTTG | CCAATT |
| | CGATTGAT | TGCGCCTC | GA |
| | AGAAAAAC | AACA | SEQ ID |
| | A | SEQ ID | NO: 16 |
| | SEQ ID | NO: 15 | |
| | NO: 14 | | |
| lukE | GATGTTGG | ATTGTTTT | CTGACT |
| | TCAAACAT | AGAATAAT | GGAAAT |
| | TAGGATAT | TAAATGAG | TACC |
| | AACATTG | CCATTGCC | SEQ ID |
| | SEQ ID | A | NO: 19 |
| | NO: 17 | SEQ ID | |
| | | NO: 18 | |
| gyrB | GGTGACTG | CTGCTTCT | CCCAGC |
| | CATTGTCA | AAACCTTC | ACCATA |
| | GATGTAAA | TAATACTT | ATTA |
| | C | GTATTTG | SEQ ID |
| | SEQ ID | SEQ ID | NO: 22 |
| | NO: 20 | NO: 21 | |

### Example 3: Characterisation of LukE and LukD Mutants

Expression, Purification of LukE and LukD

For the recombinant expression of detoxified forms of the LukE and LukD components, the lukE_stem_pET and lukD_stem_pET plasmids were generated as follows: the complete lukE gene was amplified from NCTC 8325 genomic DNA and cloned into the pET15TEV expression vector by PIPE cloning. The signal peptide was removed by whole plasmid PCR using the primers LL_56_14 SEQ ID NO: 23 (5′-TCCAGGGCAATACTAATATTGAAAATATTGGTGATGGTG-3′) and LL_57_14 SEQ ID NO: 24 (5′-TTAGTATTGCCCTGGAAGTACAGGTTTTCG-3′), generating plasmid lukE_pET. The stem loop region was removed using primers LL_08_14 SEQ ID NO: 25 (5′-AAATAGAAAGTTATGTCAGTGAAGTAGACAAGCAAAAC-3′) and LL_09_14 SEQ ID NO: 26 (5′-ACATAACTTTCTATTTTGTTTTTAGGAAGGTAATTGATTAAG-3′) and whole plasmid PCR generating plasmid lukE_stem_pET. The lukD gene lacking the signal peptide was amplified using primers LL_50_14 SEQ ID NO: 27 (5′-CTGTACTTCCAGGGCGCTCAACATATCACACCTGTAAGC-3′) and LL_51_14 SEQ ID NO: 28 (5′-ATTAAGTCGCGTTATACTCCAGGATTAGTTTCTTTAGAATCCG-3′) from NCTC 8325 genomic DNA and cloned into plasmid pET15tev using PIPE cloning, obtaining the lukD_pET construct. The primers LL_04_14 SEQ ID NO: 29 (5′-AAAATGAAGAAAGTTACAGAACTACGATTGATAGAAAAAC-3′) and LL_05_14 SEQ ID NO: 30 (5′-TAACTTTCTTCATTTTGATTTTTAGGTGCATAGTC-3′) were used to remove the stem region, through whole plasmid PCR, generating plasmid lukD_stem_pET. To improve the recombinant expression of LukD_stem protein in E. coli background, the aminoacidic sequence PSGS was inserted as linker in the stem loop devoid region. The primers LL_06_14 SEQ ID NO: 31 (5′-GAACCGTCTGGCTCTGAAAGTTACAGAACTACGATTGATAGAAAAAC-3′) and LL_07_14 SEQ ID NO: 32 (5′-TTCAGAGCCAGACGGTTCATTTTGATTTTTAGGTGCATAGTC-3′) were used to generate plasmid lukD_PSGS_pET, using the lukD_stem_pET plasmid as template. The LukD stemless variant used in the following examples includes the PSGS linker (LukD stemless).

The plasmids were transformed into Enpresso B system (Sigma Aldrich) E. coli and expression of the His-tagged proteins was induced according to the manufacturer's instructions. The recombinant proteins were purified via Ni-NTA affinity chromatography according to the manufacturer's instructions

The proteins were purified using CHAPS solubilization and TEV cleavage of the N-term His-Tag was effectuated on the Ni-NTA column, collected in the flow through and then exchanged in buffer 20 mM Bis-Tris, 150 mM NaCl, pH 6.5 for further analyses. The above mentioned LukD and LukE variants were used in the following examples and compared to the WT LukE and LukD.

Cytotoxicity Assay

The THP-1 cell line (ATCC: TIB-202) were grown as single cell suspensions in T75 cell culture flasks in RPMI supplemented with 10% of heat-inactivated FBS and passaged when culture reached a concentration between 8×105 and 1×106 cells/ml. LukED wt, LukED tagless and LukED stemless complexes were generated by mixing 20 μM of each single component and incubation for 30 minutes at room temperature. To evaluate the viability of mammalian cells after intoxication with S. aureus leukocidin complexes, THP-1 cells were plated in 96-well flat bottom tissue culture treated plates at 1×105 cells per well in RPMI supplemented with 10% of heat-inactivated FBS. Cells were intoxicated for 1 h at 37° C., 5% CO2 with serial two-fold dilutions (2 μM-0.016 μM final concentration) of LukED wt, LukED tagless and LukED stemless. After the intoxication, CellTiter (Promega) was added to each well and the cells were incubated for an additional 4 h at 37° C., 5% CO₂. The colorimetric reaction was measured with a spectrophotometer at 560/590 nm. The absorbance is directly proportional to the number of living cells in culture.

Stability of LukED Complex

The thermal stability of purified recombinant LukE and LukD protein variants was assessed by differential scanning calorimetry (DSC) using a MicroCal VP-Capillary DSC instrument (GE Healthcare) and standard operating conditions. Briefly, protein samples were prepared at a concentration of approximately 0.5 mg/mL in buffer containing 20 mM HEPES, 300 mM NaCl, pH 7.4. The DSC temperature scan ranged from 10° C. to 110° C., with a thermal ramping rate of 200° C. per hour and a 4 second filter period. Data were analysed by subtraction of the reference data for a sample containing buffer only, using the Origin 7 software (MicroCal).

### Example 4: In Vivo Activity of LukE and LukD Mutants

Mouse Skin Infection Model

Five-week old female CD1 mice (pathogen free) were immunised two times (0 and 14 days) intramuscularly (50 μl for each leg) with 10 μg of each antigen (LukE and LukD—stemless) adjuvanted with alum. Immunized mice were infected subcutaneously with a sub-lethal dose of S. aureus strain USA300 Lac (˜1-2×10⁷ CFU per mouse) 10 days after the second immunisation. Five days after the infection mice were anesthetized and photos of the skin lesions were taken with a Nikon Coolpix S9600 MP4 Land camera in order to measure the lesion area using the ImageJ-NIH software (National Institute of health, USA). Mice were sacrificed seven days after the infection and the lesion areas were collected using an 8 mm AcuPunch (biopsy punch, Acuderm Inc., USA). Each skin sample was homogenized in 2 ml PBS and serial dilutions of the homogenate were spotted in double (10 ul) on TSA plates to determine the CFU counts.

SEQUENCE LISTING
SEQ ID NO: 1
lukD Δsp (signal peptide removed),
NCTC8325 strain:
AQHITPVSEKKVDDKITLYKTTATSDNDKLNISQI
LTFNFIKDKSYDKDTLVLKAAGNINSGYKKPNPKD
YNYSQFYWGGKYNVSVSSESNDAVNVVDYAPKNQN
EEFQVQQTLGYSYGGDINISNGLSGGLNGSKSFSE
TINYKQESYRTTIDRKTNHKSIGWGVEAHKIMNNG
WGPYGRDSYDPTYGNELFLGGRQSSSNAGQNFLPT
HQMPLLARGNFNPEFISVLSHKQNDTKKSKIKVTY
QREMDRYTNQWNRLHWVGNNYKNQNTVTFTSTYEV
DWQNHTVKLIGTDSKETNPGV
lukD ΔspΔstem (signal peptide and
stem removed), NCTC8325
strain:
SEQ ID NO: 2
AQHITPVSEKKVDDKITLYKTTATSDNDKLNISQI
LTFNFIKDKSYDKDTLVLKAAGNINSGYKKPNPKD
YNYSQFYWGGKYNVSVSSESNDAVNVVDYAPKNQN
EESYRTTIDRKTNHKSIGWGVEAHKIMNNGWGPYG
RDSYDPTYGNELFLGGRQSSSNAGQNFLPTHQMPL
LARGNFNPEFISVLSHKQNDTKKSKIKVTYQREMD
RYTNQWNRLHWVGNNYKNQNTVTFTSTYEVDWQNH
TVKLIGTDSKETNPGV
lukD ΔspΔstem-PSGS (signal peptide
removed, stem replaced
with PSGS-linker), NCTC8325 strain:
SEQ ID NO: 3
AQHITPVSEKKVDDKITLYKTTATSDNDKLNISQI
LTFNFIKDKSYDKDTLVLKAAGNINSGYKKPNPKD
YNYSQFYWGGKYNVSVSSESNDAVNVVDYAPKNQN
EPSGSESYRTTIDRKTNHKSIGWGVEAHKIMNNGW
GPYGRDSYDPTYGNELFLGGRQSSSNAGQNFLPTH
QMPLLARGNFNPEFISVLSHKQNDTKKSKIKVTYQ
REMDRYTNQWNRLHWVGNNYKNQNTVTFTSTYEVD
WQNHTVKLIGTDSKETNPGV
LukE Δsp (signal peptide removed),
NCTC8325 strain:
SEQ ID NO: 4
NTNIENIGDGAEVIKRTEDVSSKKWGVTQNVQFDF
VKDKKYNKDALIVKMQGFINSRTSFSDVKGSGYEL
TKRMIWPFQYNIGLTTKDPNVSLINYLPKNKIETT
DVGQTLGYNIGGNFQSAPSIGGNGSFNYSKTISYT
QKSYVSEVDKQNSKSVKWGVKANEFVTPDGKKSAH
DRYLFVQSPNGPTGSAREYFAPDNQLPPLVQSGFN
PSFITTLSHEKGSSDTSEFEISYGRNLDITYATLF
PRTGIYAERKHNAFVNRNFVVRYEVNWKTHEIKVK
GHN
lukE ΔspΔstem (signal peptide and
stem removed), NCTC8325
strain:
SEQ ID NO: 5
NTNIENIGDGAEVIKRTEDVSSKKWGVTQNVQFDF
VKDKKYNKDALIVKMQGFINSRTSFSDVKGSGYEL
TKRMIWPFQYNIGLTTKDPNVSLINYLPKNKIESY
VSEVDKQNSKSVKWGVKANEFVTPDGKKSAHDRYL
FVQSPNGPTGSAREYFAPDNQLPPLVQSGFNPSFI
TTLSHEKGSSDTSEFEISYGRNLDITYATLFPRTG
IYAERKHNAFVNRNFVVRYEVNWKTHEIKVKGHN
lukE ΔspΔstem-PSGS (signal peptide
removed, stem replaced
with PSGS-linker), NCTC8325 strain:
SEQ ID NO: 6
NTNIENIGDGAEVIKRTEDVSSKKWGVTQNVQFDF
VKDKKYNKDALIVKMQGFINSRTSFSDVKGSGYEL
TKRMIWPFQYNIGLTTKDPNVSLINYLPKNKIEPS
GSSYVSEVDKQNSKSVKWGVKANEFVTPDGKKSAH
DRYLFVQSPNGPTGSAREYFAPDNQLPPLVQSGFN
PSFITTLSHEKGSSDTSEFEISYGRNLDITYATLF
PRTGIYAERKHNAFVNRNFVVRYEVNWKTHEIKVK
GHN
linker
SEQ ID NO: 7
PSGS
ClfA N2N3 P116S/Y118A with PglB
consensus sequence (KDQNATK,
underlined) substituted for residue 1337
SEQ ID NO: 8
VAADAPAAGTDITNQLTNVTVGIDSGTTVYPHQAG
YVKLNYGFSVPNSAVKGDTFKITVPKELNLNGVTS
TAKVPPIMAGDQVLANGVIDSDGNVIYTFTDYVNT
KDDVKATLTMSAAIDPENVKKTGNVTLATGIGSTT
ANKTVLVDYEKYGKFYNLSIKGTIDQIDKTNNTYR
QTIYVNPSGDNVIAPVLTGNLKPNTDSNALIDQQN
TSIKVYKVDNAADLSESYFVNPENFEDVTNSVNIT
FPNPNQYKVEFNTPDDQITTPYIVWNGHIDPNSKG
DLALRSTLYGYNSNIIWRSMSWDNEVAFNNGSGSG
DGIDKPVVPEQPDEPGEIEPKDQNATKPED
Mature Hla H35L/H48C/G122C with PglB
consensus sequence
(KDQNRTK) substituted for residue K131
SEQ ID NO: 9
ADSDINIKTGTTDIGSNTTVKTGDLVTYDKENGML
KKVFYSFIDDKNCNKKLLVIRTKGTIAGQYRVYSE
EGANKSGLAWPSAFKVQLQLPDNEVAQISDYYPRN
SIDTKEYMSTLTYGFNCNVTGDDTGKDQNRTKIGG
LIGANVSIGHTLKYVQPDFKTILESPTDKKVGWKV
IFNNMVNQNWGPYDRDSWNPVYGNQLFMKTRNGSM
KAADNFLDPNKASSLLSSGFSPDFATVITMDRKAS
KQQTNIDVIYERVRDDYQLHWTSTNWKGTNTKDKW
IDRSSERYKIDWEKEEMTN
SpA IgG binding portion (EDABC domains)
SEQ ID NO: 10
AQHDEAKKNAFYQVLNMPNLNADQRNGFIQSLKAA
PSQSANVLGEAQKLNDSQAPKADAKRNNFNKDKKS
AFYEILNMPNLNEAQRNGFIQSLKAAPSQSTNVLG
EAKKLNESQAPKADNNFNKEKKNAFYEILNMPNLN
EEQRNGFIQSLKAAPSQSANLLSEAKKLNESQAPK
ADNKFNKEKKNAFYEILHLPNLNEEQRNGFIQSLK
AAPSQSANLLAEAKKLNDAQAPKADNKFNKEKKNA
FYEILHLPNLTEEQRNGFIQSLKAAPSVSKEILAE
AKKLNDAQAPK
tag
SEQ ID NO: 11
HRHR
Sa_16s_+332_F primer
SEQ ID NO: 12
GAGACACGGTCCAGACTCCT
Sa_16s_+437_R primer
SEQ ID NO: 13
ACGATCCGAAGACCTTCATC
lukD Forward Primer
SEQ ID NO: 14
GAAAGTTACAGAACTACGATTGATAGAAAAACA
lukD Reverse Primer
SEQ ID NO: 15
ATTATTCATAATTTTGTGCGCCTCAACA
lukD Probe
SEQ ID NO: 16
CCCCAGCCAATTGA
lukE Forward Primer
SEQ ID NO: 17
GATGTTGGTCAAACATTAGGATATAACATTG
lukE Reverse Primer
SEQ ID NO: 18
ATTGTTTTAGAATAATTAAATGAGCCATTGCCA
lukE Probe
SEQ ID NO: 19
CTGACTGGAAATTACC
gyrB Forward Primer
SEQ ID NO: 20
GGTGACTGCATTGTCAGATGTAAAC
gyrB Reverse Primer
SEQ ID NO: 21
CTGCTTCTAAACCTTCTAATACTTGTATTTG
gyrB Probe
SEQ ID NO: 22
CCCAGCACCATAATTA
LL_56_14 primer
SEQ ID NO: 23
TCCAGGGCAATACTAATATTGAAAATATTGGTGATGGTG
LL_57_14 primer
SEQ ID NO: 24
TTAGTATTGCCCTGGAAGTACAGGTTTTCG
LL_08_14 primer
SEQ ID NO: 25
AAATAGAAAGTTATGTCAGTGAAGTAGACAAGCAAAAC
LL_09_14 primer
SEQ ID NO: 26
ACATAACTTTCTATTTTGTTTTTAGGAAGGTAATTGATTAAG
LL_50_14 primer
SEQ ID NO: 27
CTGTACTTCCAGGGCGCTCAACATATCACACCTGTAAGC
LL_51_14 primer
SEQ ID NO: 28
ATTAAGTCGCGTTATACTCCAGGATTAGTTTCTTTAGAATCCG
LL_04_14 primer
SEQ ID NO: 29
AAAATGAAGAAAGTTACAGAACTACGATTGATAGAAAAAC
LL_05_14 primer
SEQ ID NO: 30
TAACTTTCTTCATTTTGATTTTTAGGTGCATAGTC
LL_06_14 primer
SEQ ID NO: 31
GAACCGTCTGGCTCTGAAAGTTACAGAACTACGATTGAT
AGAAAAAC
LL_07_14 primer
SEQ ID NO: 32
TTCAGAGCCAGACGGTTCATTTTGATTTTTAGGTGCATAGTC

Aspects of the invention are summarised in the subsequent numbered paragraphs:

### Embodiments

1. A modified Staphylococcal leukocidin component polypeptide in which the stem domain has been fully or partially deleted, and wherein the unmodified leukocidin component polypeptide comprises or consists of an amino acid sequence having at least 80%, 85%, 90%, 92%, 95%, 96%, 97%, 98%, 99%, or 100% sequence identity to:
   a. a polypeptide comprising the amino acid sequence of SEQ ID NO.: 1; or
   b. a polypeptide comprising the amino acid sequence of SEQ ID NO.: 4.
2. The polypeptide of embodiment 1, wherein:
   a. the unmodified leukocidin component polypeptide is SEQ ID NO.: 1, and one or more of amino acids E107 to Q146 of SEQ ID NO.: 1 are deleted; or
   b. the unmodified leukocidin component polypeptide is SEQ ID NO.: 4 and one or more of amino acids T104 to K142 of SEQ ID NO.: 4 are deleted.
3. The polypeptide of any one of the preceding embodiments wherein at least 5, 10, 15, 20, 30, 40, 50 60, 70 or 90 amino acids of the unmodified leukocidin component polypeptide are deleted.
4. The polypeptide of claim 3 where the at least 5 amino acid which are deleted are consecutive.
5. The polypeptide of any one of the preceding embodiments, wherein 1 to 10 amino acids flanking the N-terminus and/or at the C-terminus of the stem domain of the unmodified leukocidin component polypeptide are deleted, for example, 1, 2, 3, 4, 5, 6, 7, 8, 9 or 10 amino acids flanking the N-terminus and/or at the C-terminus of the stem domain.
6. The polypeptide of any one of the preceding embodiments wherein where the unmodified leukocidin component polypeptide is SEQ ID NO.: 1, residue Y116 of SEQ ID NO.: 1 is deleted, and where the unmodified leukocidin component polypeptide is SEQ ID NO.: 4, residue Y113 of SEQ ID NO.: 4 is deleted.
7. The polypeptide of any one of the preceding embodiments comprising an amino acid sequence having at least 80%, 85%, 90%, 92%, 95%, 96%, 97%, 98%, 99%, or 100% sequence identity to SEQ ID NO.: 2 or SEQ ID NO.: 5.
8. The polypeptide of embodiment 6, further comprising 1, 2, 3, 4, or 5 single amino acid substitutions, deletions and/or insertions.
9. The polypeptide of any one of the preceding embodiments wherein the amino acid sequence which is deleted within the stem domain is replaced by a linker.
10. The polypeptide of embodiment 9 wherein the linker comprises or consists of a polypeptide comprising four amino acids.
11. The polypeptide of embodiment 10 wherein the linker comprises or consists of an amino acid sequence of SEQ ID NO.: 7.
12. The polypeptide of embodiment 11, comprising an amino acid sequence having at least 80%, 85%, 90%, 92%, 95%, 96%, 97%, 98%, 99%, or 100% sequence identity to SEQ ID NO.: 3 or SEQ ID NO.: 6.
13. The polypeptide of embodiment 11, further comprising 1, 2, 3, 4, or 5 single amino acid substitutions, deletions and/or insertions.
14. The polypeptide of any one of the preceding embodiments, wherein the modified leukocidin component is detoxified.
15. The polypeptide of any one of the preceding embodiments, wherein the polypeptide can elicit antibodies which bind to epitopes of a polypeptide consisting of the amino acid sequence of SEQ ID NO.: 1 or SEQ ID NO.: 4 which are maintained in the sequence of the modified leucocidin component polypeptide, and/or of complexes thereof.
16. A polynucleotide encoding the polypeptide of anyone of the preceding embodiments.
17. A vector comprising the polynucleotide of embodiment 16.
18. A host cell comprising the polynucleotide of embodiment 15 or the vector of embodiment 17.
19. A method of producing the polypeptide of any one of the preceding embodiments, said method comprising
   a. Culturing the host cell of embodiment 18 under suitable condition for the production of proteins and
   b. Isolating the polypeptide.
20. A complex comprising a modified leukocidin component polypeptide of any one of the preceding embodiments.
21. A complex comprising:
   a. a polypeptide comprising an amino acid sequence an amino acid sequence having at least 80%, 85%, 90%, 92%, 95%, 96%, 97%, 98%, 99%, or 100% sequence identity to SEQ ID NO.: 3, and/or
   b. A polypeptide comprising an amino acid sequence having at least 80%, 85%, 90%, 92%, 95%, 96%, 97%, 98%, 99%, or 100% sequence identity to SEQ ID NO.: 5.
22. The complex of embodiment 21, wherein:
   a. the polypeptide comprising an amino acid sequence an amino acid sequence having at least 80%, 85%, 90%, 92%, 95%, 96%, 97%, 98%, 99%, or 100% sequence identity to SEQ ID NO.: 3, further comprises 1, 2, 3, 4, or 5 single amino acid substitutions, deletions and/or insertions; and/or
   b. the polypeptide comprising an amino acid sequence having at least 80%, 85%, 90%, 92%, 95%, 96%, 97%, 98%, 99%, or 100% sequence identity to SEQ ID NO.: 5, further comprises 1, 2, 3, 4, or 5 single amino acid substitutions, deletions and/or insertions.
23. The complex of any one of embodiments 20-22, wherein the complex is detoxified.
24. An immunogenic composition comprising the polypeptide or the complex of any one of the preceding embodiments.
25. The immunogenic composition of any one of the preceding embodiments, further comprising:
   a. a ClfA antigen;
   b. a Hla antigen;
   c. a SpA antigen; and
   d. a staphylococcal capsular polysaccharide.
26. The immunogenic composition according to embodiment 25, wherein the composition comprises a S. aureus serotype 5 capsular polysaccharide and a type 8 capsular polysaccharide.
27. The immunogenic composition any one of embodiments 25 to 26, wherein the immunogenic composition comprises
   a. a ClfA antigen comprising an amino acid sequence having at least 80%, 85%, 90%, 92%, 95%, 96%, 97%, 98%, 99%, or 100% sequence identity to SEQ ID NO: 8;
   b. a Hla antigen comprising the amino acid sequence having at least 80%, 85%, 90%, 92%, 95%, 96%, 97%, 98%, 99%, or 100% sequence identity to SEQ ID NO: 9;
   c. a SpA antigen comprising the amino acid sequence having at least 80%, 85%, 90%, 92%, 95%, 96%, 97%, 98%, 99%, or 100% sequence identity to SEQ ID NO: 10;
   d. a S. aureus serotype 5 capsular polysaccharide; and
   e. a S. aureus serotype type 8 capsular polysaccharide.
28. The immunogenic composition according to any one of embodiments 25 to 27, wherein the capsular polysaccharide is conjugated to a carrier protein.
29. The immunogenic composition according to any one of embodiments 25 to 28, wherein the capsular polysaccharide is a S. aureus serotype 5 and/or type 8 capsular polysaccharide.
30. The immunogenic composition of any one of embodiments 25 to 29, wherein the capsular polysaccharide is conjugated to one of the antigens (a)-(c) of embodiment 25.
31. The immunogenic composition according to any one of embodiments 25 to 30, wherein the composition comprises a S. aureus serotype 5 capsular polysaccharide conjugated to a Hla antigen and/or a type 8 capsular polysaccharide conjugated to a ClfA antigen.
32. The immunogenic composition according to embodiment 31, wherein said ClfA-type 8 capsular polysaccharide and Hla-serotype 5 capsular polysaccharide conjugates are bioconjugates.
33. The immunogenic composition according to any one of the preceding embodiments, which composition comprises an adjuvant.
34. An immunogenic composition according to embodiment 33, wherein the adjuvant comprises a saponin and a lipopolysaccharide.
35. An immunogenic composition according to embodiment 34, wherein the adjuvant comprises a saponin, and a lipopolysaccharide in a liposomal formation.
36. An immunogenic composition according to embodiment 34 or embodiment 37, further comprising a sterol.
37. An immunogenic composition according to claim any one of embodiments 34 to 36, wherein the saponin is an immunologically active saponin fraction derived from the bark of Quillaja Saponaria Molina.
38. An immunogenic composition according to embodiment 37, wherein the saponin is QS21.
39. An immunogenic composition according to any one of embodiments 34 to 38, wherein the lipopolysaccharide is a lipid A derivative.
40. An immunogenic composition according to embodiment 39, wherein the lipopolysaccharide is 3D-MPL.
41. An immunogenic composition according to any one of embodiments 36 to 40, wherein the sterol is cholesterol.
42. A vaccine comprising the immunogenic composition of any one of embodiments 24 to 41.
43. A method for the treatment or prevention of Staphylococcus aureus infection in a subject in need thereof comprising administering to said subject a therapeutically effective amount of the polypeptide of any one of the preceding embodiments, the complex of any one of the preceding embodiments, the immunogenic composition of any one of the preceding embodiments, or the vaccine of any one of the preceding embodiments.
44. The method of embodiment 43 wherein the subject is a mammal.
45. The method of embodiment 44 wherein the subject is a human.
46. A method of immunising a human host against Staphylococcus aureus infection comprising administering to the host an immunoprotective dose of the polypeptide of any one of the preceding embodiments, the complex of any one of the preceding embodiments, the immunogenic composition of any one of the preceding embodiments, or the vaccine of any one of the preceding embodiments.
47. A method of inducing an immune response to Staphylococcus aureus in a subject, the method comprising administering a therapeutically or prophylactically effective amount of the polypeptide of any one of the preceding embodiments, the complex of any one of the preceding embodiments, the immunogenic composition of any one of the preceding embodiments, or the vaccine of any one of the preceding embodiments.
48. The polypeptide of any one of the preceding embodiments, the complex of any one of the preceding embodiments, the immunogenic composition of any one of the preceding embodiments, or the vaccine of any one of the preceding embodiments for use as a medicament.
49. The polypeptide of any one of the preceding embodiments, the complex of any one of the preceding embodiments, the immunogenic composition of any one of the preceding embodiments, or the vaccine of any one of the preceding embodiments, for use in the treatment or prevention of a disease caused by Staphylococcus aureus infection.
50. Use of the polypeptide of any one of the preceding embodiments, the complex of any one of the preceding embodiments, the immunogenic composition of any one of the preceding embodiments, or the vaccine of any one of the preceding embodiments, in the manufacture of a medicament for the treatment or prevention of a disease caused by Staphylococcus aureus infection.
51. A pharmaceutical for treatment or prevention of Staphylococcus aureus infection comprising the polypeptide of any one of the preceding embodiments, the complex of any one of the preceding embodiments, the immunogenic composition of any one of the preceding embodiments, or the vaccine of any one of the preceding embodiments.

1. A modified Staphylococcal leukocidin component polypeptide in which the stem domain has been at least partially deleted respective to an unmodified leukocidin component polypeptide, wherein the unmodified leukocidin component polypeptide comprises or consists of an amino acid sequence having at least 80%, 85%, 90%, 92%, 95%, 96%, 97%, 98%, 99%, or 100% sequence identity to:
a. a polypeptide comprising the amino acid sequence of SEQ ID NO.: 1; or
b. a polypeptide comprising the amino acid sequence of SEQ ID NO.: 4.

2. The polypeptide of claim 1, wherein:
a. the unmodified leukocidin component polypeptide is SEQ ID NO.: 1, and one or more of amino acids E107 to Q146 of SEQ ID NO.: 1 are deleted; or
b. the unmodified leukocidin component polypeptide is SEQ ID NO.: 4 and one or more of amino acids T104 to K142 of SEQ ID NO.: 4 are deleted,
and wherein at least 10 consecutive amino acids of the unmodified leukocidin component polypeptide are deleted.

3. The polypeptide of claim 1, wherein the stem domain has been fully deleted.

4. The polypeptide of claim 1, wherein 1 to 10 amino acids flanking the N-terminus and/or at the C-terminus of the stem domain of the unmodified leukocidin component polypeptide are deleted.

5. The polypeptide of claim 1 comprising an amino acid sequence having at least 80%, 85%, 90%, 92%, 95%, 96%, 97%, 98%, 99%, or 100% sequence identity to SEQ ID NO.: 2 or SEQ ID NO.: 5.

6. The polypeptide of claim 1 wherein the amino acid sequence which is deleted within the stem domain is replaced by a linker.

7. The polypeptide of claim 5, comprising an amino acid sequence having at least 80%, 85%, 90%, 92%, 95%, 96%, 97%, 98%, 99%, or 100% sequence identity to SEQ ID NO.: 3 or SEQ ID NO.: 6.

8. The polypeptide of claim 1, wherein the modified leukocidin component is detoxified.

9. The polypeptide of claim 1, wherein the polypeptide can elicit antibodies which bind to epitopes of a polypeptide consisting of the amino acid sequence of SEQ ID NO.: 1 or SEQ ID NO.: 4 which are maintained in the sequence of the modified leucocidin component polypeptide, and/or of complexes thereof.

10. A polynucleotide encoding the polypeptide of claim 1.

11. A complex comprising a modified leukocidin component polypeptide of claim 1.

12. A complex comprising:
a. a polypeptide comprising an amino acid sequence an amino acid sequence having at least 80%, 85%, 90%, 92%, 95%, 96%, 97%, 98%, 99%, or 100% sequence identity to SEQ ID NO.: 3, and/or
b. A polypeptide comprising an amino acid sequence having at least 80%, 85%, 90%, 92%, 95%, 96%, 97%, 98%, 99%, or 100% sequence identity to SEQ ID NO.: 5.

13. An immunogenic composition comprising the polypeptide of claim 1.

14. The immunogenic composition of claim 13, further comprising:
a. a ClfA antigen;
b. a Hla antigen;
c. a SpA antigen; and
d. a staphylococcal capsular polysaccharide.

15. A vaccine comprising the immunogenic composition of claim 13.

16. (canceled)

17. (canceled)

18. (canceled)

19. An immunogenic composition comprising the complex of claim 11.

20. A method of inducing an immune response to Staphylococcus aureus in a subject, the method comprising administering a therapeutically or prophylactically effective amount of the polypeptide of claim 1.

21. A method of treating and/or preventing Staphylococcus aureus infections in a subject comprising administering a therapeutically or prophylactically effective amount of the polypeptide of claim 1.

22. A method of inducing an immune response to Staphylococcus aureus in a subject, the method comprising administering a therapeutically or prophylactically effective amount of the complex of claim 11.

23. A method of treating and/or preventing Staphylococcus aureus infections in a subject comprising administering a therapeutically or prophylactically effective amount of the complex of claim 11.


SEQUENCE LISTING (32 sequences)

SEQ ID NO: 1 - protein, 301 aa, Artificial
  lukD (Delta)sp (signal peptide removed)
AQHITPVSEKKVDDKITLYKTTATSDNDKLNISQILTFNFIKDKSYDKDTLVLKAAGNINSGYKKPNPKDYNYSQFYWGGKYNVSVSSESNDAVNVVDYAPKNQNEEFQVQQTLGYSYGGDINISNGLSGGLNGSKSFSETINYKQESYRTTIDRKTNHKSIGWGVEAHKIMNNGWGPYGRDSYDPTYGNELFLGGRQSSSNAGQNFLPTHQMPLLARGNFNPEFISVLSHKQNDTKKSKIKVTYQREMDRYTNQWNRLHWVGNNYKNQNTVTFTSTYEVDWQNHTVKLIGTDSKETNPGV

SEQ ID NO: 2 - protein, 261 aa, Artificial
  lukD (Delta)sp(Delta)stem (signal peptide and stem removed)
AQHITPVSEKKVDDKITLYKTTATSDNDKLNISQILTFNFIKDKSYDKDTLVLKAAGNINSGYKKPNPKDYNYSQFYWGGKYNVSVSSESNDAVNVVDYAPKNQNEESYRTTIDRKTNHKSIGWGVEAHKIMNNGWGPYGRDSYDPTYGNELFLGGRQSSSNAGQNFLPTHQMPLLARGNFNPEFISVLSHKQNDTKKSKIKVTYQREMDRYTNQWNRLHWVGNNYKNQNTVTFTSTYEVDWQNHTVKLIGTDSKETNPGV

SEQ ID NO: 3 - protein, 265 aa, Artificial
  lukD Delta)sp(Delta)stem-PSGS (signal peptide removed, stem replaced with PSGS-linker)
AQHITPVSEKKVDDKITLYKTTATSDNDKLNISQILTFNFIKDKSYDKDTLVLKAAGNINSGYKKPNPKDYNYSQFYWGGKYNVSVSSESNDAVNVVDYAPKNQNEPSGSESYRTTIDRKTNHKSIGWGVEAHKIMNNGWGPYGRDSYDPTYGNELFLGGRQSSSNAGQNFLPTHQMPLLARGNFNPEFISVLSHKQNDTKKSKIKVTYQREMDRYTNQWNRLHWVGNNYKNQNTVTFTSTYEVDWQNHTVKLIGTDSKETNPGV

SEQ ID NO: 4 - protein, 283 aa, Artificial
  LukE (Delta)sp (signal peptide removed)
NTNIENIGDGAEVIKRTEDVSSKKWGVTQNVQFDFVKDKKYNKDALIVKMQGFINSRTSFSDVKGSGYELTKRMIWPFQYNIGLTTKDPNVSLINYLPKNKIETTDVGQTLGYNIGGNFQSAPSIGGNGSFNYSKTISYTQKSYVSEVDKQNSKSVKWGVKANEFVTPDGKKSAHDRYLFVQSPNGPTGSAREYFAPDNQLPPLVQSGFNPSFITTLSHEKGSSDTSEFEISYGRNLDITYATLFPRTGIYAERKHNAFVNRNFVVRYEVNWKTHEIKVKGHN

SEQ ID NO: 5 - protein, 244 aa, Artificial
  lukE (Delta)sp(Delta)stem (signal peptide and stem removed)
NTNIENIGDGAEVIKRTEDVSSKKWGVTQNVQFDFVKDKKYNKDALIVKMQGFINSRTSFSDVKGSGYELTKRMIWPFQYNIGLTTKDPNVSLINYLPKNKIESYVSEVDKQNSKSVKWGVKANEFVTPDGKKSAHDRYLFVQSPNGPTGSAREYFAPDNQLPPLVQSGFNPSFITTLSHEKGSSDTSEFEISYGRNLDITYATLFPRTGIYAERKHNAFVNRNFVVRYEVNWKTHEIKVKGHN

SEQ ID NO: 6 - protein, 248 aa, Artificial
  lukE (Delta)sp(Delta)stem-PSGS (signal peptide removed, stem replaced with PSGS-linker)
NTNIENIGDGAEVIKRTEDVSSKKWGVTQNVQFDFVKDKKYNKDALIVKMQGFINSRTSFSDVKGSGYELTKRMIWPFQYNIGLTTKDPNVSLINYLPKNKIEPSGSSYVSEVDKQNSKSVKWGVKANEFVTPDGKKSAHDRYLFVQSPNGPTGSAREYFAPDNQLPPLVQSGFNPSFITTLSHEKGSSDTSEFEISYGRNLDITYATLFPRTGIYAERKHNAFVNRNFVVRYEVNWKTHEIKVKGHN

SEQ ID NO: 7 - protein, 4 aa, Artificial
  Linker
PSGS

SEQ ID NO: 8 - protein, 346 aa, Artificial
  ClfA N2N3 P116S/Y118A  with PglB consensus sequence (KDQNATK,) substituted for residue I337
VAADAPAAGTDITNQLTNVTVGIDSGTTVYPHQAGYVKLNYGFSVPNSAVKGDTFKITVPKELNLNGVTSTAKVPPIMAGDQVLANGVIDSDGNVIYTFTDYVNTKDDVKATLTMSAAIDPENVKKTGNVTLATGIGSTTANKTVLVDYEKYGKFYNLSIKGTIDQIDKTNNTYRQTIYVNPSGDNVIAPVLTGNLKPNTDSNALIDQQNTSIKVYKVDNAADLSESYFVNPENFEDVTNSVNITFPNPNQYKVEFNTPDDQITTPYIVVVNGHIDPNSKGDLALRSTLYGYNSNIIWRSMSWDNEVAFNNGSGSGDGIDKPVVPEQPDEPGEIEPKDQNATKPED

SEQ ID NO: 9 - protein, 299 aa, Artificial
  Mature Hla H35L/H48C/G122C with PglB consensus sequence (KDQNRTK) substituted for residue K131
ADSDINIKTGTTDIGSNTTVKTGDLVTYDKENGMLKKVFYSFIDDKNCNKKLLVIRTKGTIAGQYRVYSEEGANKSGLAWPSAFKVQLQLPDNEVAQISDYYPRNSIDTKEYMSTLTYGFNCNVTGDDTGKDQNRTKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWIDRSSERYKIDWEKEEMTN

SEQ ID NO: 10 - protein, 291 aa, Artificial
  SpA IgG binding portion
AQHDEAKKNAFYQVLNMPNLNADQRNGFIQSLKAAPSQSANVLGEAQKLNDSQAPKADAKRNNFNKDKKSAFYEILNMPNLNEAQRNGFIQSLKAAPSQSTNVLGEAKKLNESQAPKADNNFNKEKKNAFYEILNMPNLNEEQRNGFIQSLKAAPSQSANLLSEAKKLNESQAPKADNKFNKEKKNAFYEILHLPNLNEEQRNGFIQSLKAAPSQSANLLAEAKKLNDAQAPKADNKFNKEKKNAFYEILHLPNLTEEQRNGFIQSLKAAPSVSKEILAEAKKLNDAQAPK

SEQ ID NO: 11 - protein, 4 aa, Artificial
  tag
HRHR

SEQ ID NO: 12 - DNA, 20 bp, Artificial
  primer
gagacacggtccagactcct

SEQ ID NO: 13 - DNA, 20 bp, Artificial
  Primer
acgatccgaagaccttcatc

SEQ ID NO: 14 - DNA, 33 bp, Artificial
  lukD Forward Primer
gaaagttacagaactacgattgatagaaaaaca

SEQ ID NO: 15 - DNA, 28 bp, Artificial
  lukD Reverse Primer
attattcataattttgtgcgcctcaaca

SEQ ID NO: 16 - DNA, 14 bp, Artificial
  lukD Probe
ccccagccaattga

SEQ ID NO: 17 - DNA, 31 bp, Artificial
  lukE Forward Primer
gatgttggtcaaacattaggatataacattg

SEQ ID NO: 18 - DNA, 33 bp, Artificial
  lukE Reverse Primer
attgttttagaataattaaatgagccattgcca

SEQ ID NO: 19 - DNA, 16 bp, Artificial
  lukE Probe
ctgactggaaattacc

SEQ ID NO: 20 - DNA, 25 bp, Artificial
  gyrB Forward Primer
ggtgactgcattgtcagatgtaaac

SEQ ID NO: 21 - DNA, 31 bp, Artificial
  gyrB Reverse Primer
ctgcttctaaaccttctaatacttgtatttg

SEQ ID NO: 22 - DNA, 16 bp, Artificial
  gyrB Probe
cccagcaccataatta

SEQ ID NO: 23 - DNA, 39 bp, Artificial
  Primer
tccagggcaatactaatattgaaaatattggtgatggtg

SEQ ID NO: 24 - DNA, 30 bp, Artificial
  Primer
ttagtattgccctggaagtacaggttttcg

SEQ ID NO: 25 - DNA, 38 bp, Artificial
  Primer
aaatagaaagttatgtcagtgaagtagacaagcaaaac

SEQ ID NO: 26 - DNA, 42 bp, Artificial
  Primer
acataactttctattttgtttttaggaaggtaattgattaag

SEQ ID NO: 27 - DNA, 39 bp, Artificial
  Primer
ctgtacttccagggcgctcaacatatcacacctgtaagc

SEQ ID NO: 28 - DNA, 43 bp, Artificial
  Primer
attaagtcgcgttatactccaggattagtttctttagaatccg

SEQ ID NO: 29 - DNA, 40 bp, Artificial
  Primer
aaaatgaagaaagttacagaactacgattgatagaaaaac

SEQ ID NO: 30 - DNA, 35 bp, Artificial
  Primer
taactttcttcattttgatttttaggtgcatagtc

SEQ ID NO: 31 - DNA, 47 bp, Artificial
  Primer
gaaccgtctggctctgaaagttacagaactacgattgatagaaaaac

SEQ ID NO: 32 - DNA, 42 bp, Artificial
  Primer
ttcagagccagacggttcattttgatttttaggtgcatagtc